(19) United States
(12) Patent Application Publication
(10) Pub. No.: US 2025/0281414 A1
(43) Pub. Date: Sep. 11, 2025

(54) HIGH DOSE ENDOXIFEN FORMULATIONS AND METHODS OF USE

(71) Applicant: Atossa Therapeutics, Inc., Seattle, WA (US)

(72) Inventor: Steven C. QUAY, Seattle, WA (US)

(21) Appl. No.: 18/859,140

(22) PCT Filed: Apr. 25, 2023
(86) PCT No.: PCT/US2023/019814

§ 371 Date: Oct. 22, 2024

Related U.S. Application Data
(60) Provisional application No. 63/334,845, filed on Apr. 26, 2022.
(60) Provisional application No. 63/437,048, filed on Jan. 4, 2023.

(51) Int. Cl. A61K 9/48; A61K 9/00; A61K 31/138

(52) U.S. Cl. CPC A61K 9/4866; A61K 9/0002; A61K 9/4816; A61K 9/4858; A61K 31/138

(57) ABSTRACT

Described herein are pharmaceutical compositions comprising high doses of (Z)-endoxifen. A high dose endoxifen composition may be formulated to prevent crosslinking between the endoxifen active pharmaceutical ingredient and a surrounding layer, such as a capsule. The compositions may be further formulated as enteric resistant compositions for oral administration and delivery to an intestine of a subject. Also described herein are methods of treatment using high dose endoxifen compositions.

### CROSS-REFERENCE

The present application claims the benefit of U.S. Provisional Application No. 63/334,845, entitled “HIGH DOSE ENDOXIFEN FORMULATIONS AND METHODS OF USE,” filed on Apr. 26, 2022, and U.S. Provisional Application No. 63/437,048, entitled “HIGH DOSE ENDOXIFEN FORMULATIONS AND METHODS OF USE,” filed on Jan. 4, 2023, which applications are herein incorporated by reference in their entireties for all purposes.

### BACKGROUND

While cancer survival rates have been increasing over the last few decades, cancer remains the second leading cause of death in the United States. Despite extensive research into treatments and prevention, drug resistance and relapse remain limiting factors in developing cancer cures. To combat these and other factors, there is a need for cancer therapies capable of effectively killing cancer cells, overcoming drug resistance, and preventing relapse.

### SUMMARY

In various aspects, the present disclosure provides a composition comprising: a drug formulation comprising: not less than 4% (Z)-endoxifen by weight, and not less than 1% croscarmellose sodium by weight; and an enteric-resistant delayed release capsule encapsulating the drug formulation.

In some aspects, a percent of (Z)-endoxifen with respect to total endoxifen in the drug formulation is not less than 95%, not less than 97%, or not less than 98%, wherein the total endoxifen consists of (Z)-endoxifen and (E)-endoxifen. In some aspects, the percent of (Z)-endoxifen with respect to total endoxifen in the drug formulation is not more than 100%. In some aspects, the drug formulation comprises not less than 5%, not less than 7%, not less than 10%, not less than 12%, or not less than 15% (Z)-endoxifen by weight. In some aspects, the drug formulation comprises not more than 20%, not more than 22%, not more than 25%, not more than 30%, or not more than 40% (Z)-endoxifen by weight. In some aspects, the drug formulation comprises not less than 5% and not more than 40% (Z)-endoxifen by weight. In some aspects, the drug formulation comprises not less than 10% and not more than 25% (Z)-endoxifen by weight. In some aspects, the drug formulation comprises not less than 15% and not more than 20% (Z)-endoxifen by weight. In some aspects, the drug formulation comprises not less than 17% and not more than 19% (Z)-endoxifen by weight.

In some aspects, the drug formulation comprises not less than 1 mg and not more than 80 mg (Z)-endoxifen per enteric-resistant delayed release capsule. In some aspects, the drug formulation comprises not less than 10 mg and not more than 80 mg (Z)-endoxifen per enteric-resistant delayed release capsule. In some aspects, the drug formulation comprises not less than 30 mg and not more than 50 mg (Z)-endoxifen per enteric-resistant delayed release capsule. In some aspects, the drug formulation comprises not less than 38 mg and not more than 42 mg (Z)-endoxifen per enteric-resistant delayed release capsule. In some aspects, the drug formulation comprises about 1 mg, about 2 mg, about 4 mg, about 10 mg, about 20 mg, about 40 mg, or about 80 mg (Z)-endoxifen per enteric-resistant delayed release capsule.

In some aspects, the drug formulation comprises not less than 1.5%, not less than 1.7%, not less than 2%, not less than 2.2%, not less than 2.5%, not less than 2.7%, or not less than 2.8% croscarmellose sodium by weight. In some aspects, the drug formulation comprises not more than 3%, not more than 3.2%, not more than 3.5%, not more than 4%, not more than 4.5%, or not more than 5% croscarmellose sodium by weight. In some aspects, the drug formulation comprises not less than 1% and not more than 5% croscarmellose sodium by weight. In some aspects, the drug formulation comprises not less than 2% and not more than 4% croscarmellose sodium by weight. In some aspects, the drug formulation comprises not less than 2.5% and not more than 3% croscarmellose sodium by weight. In some aspects, the drug formulation comprises not less than 2.8% and not more than 3% croscarmellose sodium by weight.

In some aspects, the drug formulation further comprises microcrystalline cellulose. In some aspects, the drug formulation comprises not less than 60%, not less than 65%, not less than 70%, not less than 75%, or not less than 77% microcrystalline cellulose by weight. In some aspects, the drug formulation comprises not more than 79%, not more than 80%, not more than 85%, not more than 90%, or not more than 95% microcrystalline cellulose by weight. In some aspects, the drug formulation comprises not less than 60% and not more than 95% microcrystalline cellulose by weight. In some aspects, the drug formulation comprises not less than 70% and not more than 90% microcrystalline cellulose by weight. In some aspects, the drug formulation comprises not less than 75% and not more than 80% microcrystalline cellulose by weight.

In some aspects, the drug formulation further comprises magnesium stearate. In some aspects, the drug formulation comprises not less than 0.3%, not less than 0.5%, not less than 0.7%, not less than 0.8%, or not less than 0.9% magnesium stearate by weight. In some aspects, the drug formulation comprises not more than 1.1%, not more than 1.2%, not more than 1.5%, not more than 1.8%, not more than 2%, or not more than 3% magnesium stearate by weight. In some aspects, the drug formulation comprises not less than 0.5% and not more than 3% magnesium stearate by weight. In some aspects, the drug formulation comprises not less than 0.5% and not more than 2% magnesium stearate by weight. In some aspects, the drug formulation comprises not less than 0.5% and not more than 1.5% magnesium stearate by weight.

In some aspects, the enteric-resistant delayed release capsule comprises hydroxypropyl methylcellulose, gellan gum, gelatin, hydroxypropyl methylcellulose phthalate, a coloring agent, an opacifier, or any combination thereof. In some aspects, the enteric-resistant delayed release capsule comprises hydroxypropyl methylcellulose. In some aspects, the enteric-resistant delayed release capsule comprises not less than 85% and not more than 97% hydroxypropyl methylcellulose by weight. In some aspects, the enteric-resistant delayed release capsule comprises gellan gum, gelatin, or a combination thereof. In some aspects, the enteric-resistant delayed release capsule comprises not less than 3% and not more than 10% gellan gum, gelatin, or the combination thereof by weight.

In some aspects, the (Z)-endoxifen comprises a polymorphic form of endoxifen.

In some aspects, the polymorphic form of endoxifen is Form I, characterized by an x-ray powder diffraction pattern comprising major peaks at 16.8±0.3°, 17.1±0.3° and 21.8±0.3° two theta. In some aspects, the x-ray powder diffraction pattern further comprises: a) at least one peak selected from 16.0±0.3°, 18.8±0.3° and 26.5±0.3° two theta; b) at least one peak selected from 12.3±0.3°, 28.0±0.3° and 29.0±0.3° two theta; or c) a combination thereof. In some aspects, the x-ray powder diffraction pattern further comprises at least one peak, or at least two peaks, at least three peaks selected from the group consisting of 16.0±0.3°, 18.8±0.3° and 26.5±0.3° two theta. In some aspects, the x-ray powder diffraction pattern further comprises at least one peak, or at least two peaks, at least three peaks selected from the group consisting of 12.3±0.3°, 28.0±0.3° and 29.0±0.3° two theta.

In some aspects, the polymorphic form of endoxifen is Form II, characterized by an x-ray powder diffraction pattern comprising major peaks at 7.0±0.3°, 11.9±0.3°, and 14.0±0.3°. In some aspects, the x-ray powder diffraction pattern further comprises at least one peak, or at least two peaks, at least three peaks, or at least four peaks selected from the group consisting of the group consisting of 18.4±0.3°, 22.0±0.3°, 6.6±0.3°, and 13.3±0.3° two theta. In some aspects, the x-ray powder diffraction pattern further comprises at least one peak, or at least two peaks, at least three peaks, at least four peaks, or at least five peaks selected from the group consisting of 20.0±0.3°, 6.6±0.3°, 13.3±0.3°, 20.0±0.3° and 22.0±0.3° two theta.

In some aspects, the polymorphic form of endoxifen is Form III, characterized by an x-ray powder diffraction pattern comprising major peaks at 11.9±0.3°, 13.9±0.3°, and 17.1±0.3° two theta. In some aspects, the x-ray powder diffraction pattern further comprises at least one peak, or at least two peaks, at least three peaks, or at least four peaks selected from the group consisting of 17.7±0.3°, 25.3±0.3°, 18.2±0.3°, and 22.5±0.3° two theta. In some aspects, the x-ray powder diffraction pattern further comprises at least one peak, or at least two peaks, at least three peaks, at least four peaks, or at least five peaks selected from the group consisting of 26.8±0.3°, 18.2±0.3°, 22.5±0.3°, 25.3±0.3° and 26.8±0.3° two theta.

In some aspects, the polymorphic form of endoxifen is Form IV, characterized by an x-ray powder diffraction pattern comprising major peaks at 4.7±0.3° two theta, 23.3±0.3°, and 13.6±0.3° two theta. In some aspects, the x-ray powder diffraction pattern further comprises at least one peak, at least two peaks, at least three peaks, or at least four peaks selected from the group consisting of 23.8±0.3°, 14.2±0.3°, 22.5±0.3°, or 15.7±0.3° two theta. In some aspects, the x-ray powder diffraction pattern further comprises at least one peak, at least two peaks, or at least three peaks selected from the group consisting of 7.1±0.3°, 20.2±0.3°, or 9.5±0.3° two theta.

In some aspects, the polymorphic form of endoxifen is Form V, characterized by an x-ray powder diffraction pattern comprising major peaks at 12.5±0.3°, 19.6±0.3°, and 8.9±0.3° two theta. In some aspects, the x-ray powder diffraction pattern further comprises at least one peak, at least two peaks, at least three peaks, or at least four peaks selected from the group consisting of 21.7±0.3°, 20.8±0.3°, 19.8±0.3°, or 16.0±0.3° two theta. In some aspects, the x-ray powder diffraction pattern further comprises at least one peak, comprises at least two peaks, at least three peaks, at least four peaks, at least five peaks, or at least six peaks selected from the group consisting of 21.7±0.3°, 20.8±0.3°, 19.8±0.3°, 16.0±0.3°, 22.0±0.3°, 13.5±0.3°, and 14.4±0.3° two theta.

In some aspects, the polymorphic form of endoxifen is Form VI, characterized by an x-ray powder diffraction pattern comprising major peaks at 9.9±0.3°, 13.4±0.3°, and 13.7±0.3° two theta. In some aspects, the x-ray powder diffraction pattern further comprises at least one peak, at least two peaks, at least three peaks, or at least four peaks selected from the group consisting of 17.6±0.3°, 18.6±0.3°, 17.3±0.3°, or 21.8±0.3° two theta. In some aspects, the x-ray powder diffraction pattern further comprises at least one peak, at least two peaks, or at least three peaks selected from the group consisting of 10.2±0.3°, 19.5±0.3°, or 14.2±0.3° two theta.

In some aspects, the polymorphic form of endoxifen is Form VII, characterized by an x-ray powder diffraction pattern comprising major peaks at 20.0±0.3°, 22.6±0.3°, and 10.6±0.3° two theta. In some aspects, the x-ray powder diffraction pattern further comprises at least one peak, at least two peaks, at least three peaks, or at least four peaks selected from the group consisting of 11.4±0.3°, 16.4±0.3°, 9.6±0.3°, or 13.3±0.3° two theta. In some aspects, the x-ray powder diffraction pattern further comprises at least one peak, at least two peaks, or at least three peaks selected from the group consisting of 18.2±0.3°, 13.1±0.3°, or 27.0±0.3° two theta.

In some aspects, the polymorphic form of endoxifen is Form VIII, characterized by an x-ray powder diffraction pattern comprising major peaks at 4.8±0.3°, 18.9±0.3°, and 9.5±0.3° two theta. In some aspects, the x-ray powder diffraction pattern further comprises at least one peak, at least two peaks, at least three peaks, or at least four peaks selected from the group consisting of 23.7±0.3°, 21.9±0.3°, 21.2±0.3°, or 12.9±0.3° two theta. In some aspects, the x-ray powder diffraction pattern further comprises at least one peak, at least two peaks, or at least three peaks selected from the group consisting of 25.0±0.3°, 21.5±0.3°, or 16.4±0.3° two theta.

In some aspects, the polymorphic form of endoxifen is Form IX, characterized by an x-ray powder diffraction pattern comprising major peaks at 19.0±0.3°, 12.9±0.3°, and 15.9±0.3° two theta. In some aspects, the x-ray powder diffraction pattern further comprises at least two peaks, at least three peaks, or at least four peaks selected from the group consisting of 21.7±0.3°, 20.8±0.3°, 21.1±0.3°, or 8.9±0.3° two theta. In some aspects, the x-ray powder diffraction pattern further comprises at least one peak, at least two peaks, or at least three peaks selected from the group consisting of 16.4±0.3°, 4.2±0.3°, or 12.7±0.3° two theta.

In some aspects, the polymorphic form of endoxifen is Form X, characterized by an x-ray powder diffraction pattern comprising major peaks at 7.2±0.3°, 14.3±0.3°, 18.7±0.3°, and two theta. In some aspects, the x-ray powder diffraction pattern further comprises at least one peak, at least two peaks, or at least three peaks selected from the group consisting of 21.5±0.3°, and 22.7±0.3°, and 17.1±0.3° two theta. In some aspects, the x-ray powder diffraction pattern further comprises at least one peak, at least two peaks, or at least three peaks selected from the group consisting of 21.8±0.3°, 27.3±0.3°, or 29.4±0.3° two theta.

In some aspects, the polymorphic form of endoxifen is Form XI, characterized by an x-ray powder diffraction pattern comprising major peaks at 14.0±0.3°, 17.7±0.3°, and 11.9±0.3° two theta. In some aspects, the x-ray powder diffraction pattern further comprises at least one peak, at least two peaks, or at least three peaks selected from the group consisting of 18.4±0.3°, 23.9±0.3°, or 17.3±0.3° two theta. In some aspects, the x-ray powder diffraction pattern further comprises at least one peak, at least two peaks, at least three peaks, or at least four peaks selected from the group consisting of 21.8±0.3°, 20.8±0.3°, and 23.0±0.3°, or 22.2±0.3° two theta.

In some aspects, the polymorphic form of endoxifen is Form XII, characterized by an x-ray powder diffraction pattern comprising major peaks at 12.5±0.3°, 15.6±0.3°, and 19.0±0.3° two theta. In some aspects, the x-ray powder diffraction pattern further comprises at least one peak, at least two peaks, at least three peaks, or at least four peaks selected from the group consisting of 21.9±0.3°, 20.2±0.3°, 16.0±0.3°, or 21.6±0.3° two theta. In some aspects, the x-ray powder diffraction pattern further comprises at least one peak, at least two peaks, or at least three peaks selected from the group consisting of 22.4±0.3°, 16.8±0.3°, or 12.8±0.3° two theta.

In some aspects, the polymorphic form of endoxifen is Form XIV, characterized by an x-ray powder diffraction pattern comprising major peaks at 11.6±0.3°, 21.3±0.3°, and 19.3±0.3° two theta. In some aspects, the x-ray powder diffraction pattern further comprises at least one peak, at least two peaks, at least three peaks, or at least four peaks selected from the group consisting of 17.5±0.3°, 15.4±0.3°, 21.6±0.3°, or 5.8±0.3° two theta. In some aspects, the x-ray powder diffraction pattern further comprises at least one peak, at least two peaks, or at least three peaks selected from the group consisting of 16.3±0.3°, 21.9±0.3°, or 23.9±0.3° two theta.

In some aspects, the polymorphic form of endoxifen is Form XV, characterized by an x-ray powder diffraction pattern comprising major peaks at 9.8±0.3°, 4.7±0.3°, and 14.0±0.3° two theta. In some aspects, the x-ray powder diffraction pattern further comprises at least one peak, at least two peaks, at least three peaks, or at least four peaks selected from the group consisting of 20.2±0.3°, 7.1±0.3°, 23.4±0.3°, or 22.4±0.3° two theta. In some aspects, the x-ray powder diffraction pattern further comprises at least one peak, at least two peaks, or at least three peaks selected from the group consisting of 21.7±0.3°, 22.7±0.3°, or 18.8±0.3° two theta.

In some aspects, the polymorphic form of endoxifen is Form XIX, characterized by an x-ray powder diffraction pattern comprising major peaks at 4.7±0.3°, 23.6±0.3°, and 18.9±0.3° two theta. In some aspects, the x-ray powder diffraction pattern further comprises at least one peak, at least two peaks, at least three peaks, or at least four peaks selected from the group consisting of 9.4±0.3°, 23.3±0.3°, 22.3±0.3°, or 20.1±0.3° two theta. In some aspects, the x-ray powder diffraction pattern further comprises at least one peak, at least two peaks, or at least three peaks selected from the group consisting of 19.6±0.3°, 7.1±0.3°, or 15.7±0.3° two theta.

In some aspects, at least 90% of the (Z)-endoxifen by weight is the polymorphic form of endoxifen.

In some aspects, the composition comprises an in vitro dissolution profile wherein: a) not more than 20% of the (Z)-endoxifen is released within 2 hours after the composition is introduced into an acidic stage of an in vitro dissolution assay; b) not less than 70% of the (Z)-endoxifen is released within 1.5 hours after the composition is introduced into a buffer stage of the in vitro dissolution assay; or c) a combination thereof. In some aspects, not more than 5%, not more than 10%, or not more than 15% of the (Z)-endoxifen is released within 2 hours after the composition is introduced into the acidic stage of the in vitro dissolution assay. In some aspects, not less than 75%, not less than 80%, or not less than 85% of the (Z)-endoxifen is released within 1.5 hours after the composition is introduced into the buffer stage of the in vitro dissolution assay. In some aspects, the buffer stage comprises a pH of less than 2. In some aspects, the buffer stage comprises a pH of about 6.8. In some aspects, the buffer stage comprises 0.75% polysorbate 80. In some aspects, upon oral administration of the composition to a subject not more than 5%, not more than 10%, not more than 15%, or not more than 20% of the (Z)-endoxifen is released in a stomach of the subject, and not less than 70%, not less than 75%, not less than 80%, or not less than 85% of the (Z)-endoxifen is released in an intestine of the subject.

In various aspects, the present disclosure provides a composition comprising: a drug formulation comprising: not less than 15% and not more than 20% (Z)-endoxifen by weight, not less than 2% and not more than 4% croscarmellose sodium by weight, not less than 0.5% and not more than 2% magnesium stearate by weight, and not less than 70% and not more than 80% microcrystalline cellulose by weight; and an enteric-resistant delayed release capsule encapsulating the drug formulation, wherein the enteric-resistant delayed release capsule comprises: not less than 85% and not more than 97% hydroxypropyl methylcellulose by weight, and not less than 3% and not more than 7% gellan gum by weight.

In some aspects, the drug formulation comprises not less than 17% and not more than 19% (Z)-endoxifen by weight. In some aspects, the drug formulation comprises not less than 2.8% and not more than 3% croscarmellose sodium by weight. In some aspects, the drug formulation comprises not less than 0.7% and not more than 1.2% magnesium stearate by weight. In some aspects, the drug formulation comprises not less than 75% and not more than 80% microcrystalline cellulose by weight.

In various aspects, the present disclosure provides a method of treating a disorder in a subject, the method comprising orally administering to the subject a composition comprising: a drug formulation comprising: not less than 4% (Z)-endoxifen by weight, and not less than 1% croscarmellose sodium by weight; and an enteric-resistant delayed release capsule encapsulating the drug formulation thereby treating the cancer.

In various aspects, the present disclosure provides a method of treating a disorder in a subject, the method comprising orally administering to the subject a composition as described herein, thereby treating the disorder.

In some aspects, the disorder is a cancer, a hormone-dependent breast disorder, or a hormone-dependent reproductive tract disorder. In some aspects, the cancer is breast cancer, cervical cancer, ovarian cancer, endometrial cancer, uterine cancer, vaginal cancer, vulvar cancer, melanoma, colorectal cancer, gastric cancer, neuroblastoma, pancreatic cancer, esophageal cancer, rectal cancer, or cholangiocarcinoma. In some aspects, the breast cancer is triple negative breast cancer, ductal carcinoma in situ, lobular carcinoma in situ, invasive ductal carcinoma, or invasive lobular carcinoma. In some aspects, the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, precocious puberty, or McCune-Albright Syndrome. In some aspects, the disorder is tamoxifen-resistant or tamoxifen-refractory.

In some aspects, the method further comprises releasing not more than 5%, not more than 10%, not more than 15%, or not more than 20% of the (Z)-endoxifen is released in a stomach of the subject, and not less than 70%, not less than 75%, not less than 80%, or not less than 85% of the (Z)-endoxifen is released in an intestine of the subject following oral administration of the composition. In some aspects, the method further comprises producing in the subject a blood plasma concentration of endoxifen that is not less than 70%, not less than 75%, not less than 80%, or not less than 85% in the (Z)-isoform.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows an HPLC chromatogram of a 40 mg (Z)-endoxifen formulation containing 40 mg (Z)-endoxifen, 173.78 mg microcrystalline cellulose, 6.50 mg croscarmellose sodium, and 2.23 mg magnesium stearate encapsulated in an enteric-resistant delayed release capsule and dissolved in a diluent to a concentration of to 0.5 mg/mL (Z)-endoxifen.

FIG. 1B shows an HPLC chromatogram of a 0.5 mg/mL (Z)-endoxifen reference sample encapsulated in an enteric-resistant delayed release capsule and dissolved in diluent.

FIG. 1C shows an HPLC chromatogram of the diluent used to dissolve the samples assayed in FIG. 1A and FIG. 1B.

FIG. 2A shows an X-ray powder diffraction pattern obtained from a sample of Form I of endoxifen.

FIG. 2B shows an X-ray powder diffraction (XRPD) pattern obtained from a sample of Form I of endoxifen.

FIG. 2C shows an XRPD pattern obtained from a sample of Form I of endoxifen.

FIG. 3 shows an XRPD pattern obtained from a sample of Form II of endoxifen.

FIG. 4 shows an XRPD pattern obtained from a sample of Form III of endoxifen.

FIG. 5 shows an XRPD pattern obtained from a sample of Form IV of endoxifen.

FIG. 6 shows an XRPD pattern obtained from a sample of Form V of endoxifen.

FIG. 7 shows an XRPD pattern obtained from a sample of Form VI of endoxifen.

FIG. 8 shows an XRPD pattern obtained from a sample of Form VII of endoxifen.

FIG. 9 shows an XRPD pattern obtained from a sample of Form VIII of endoxifen.

FIG. 10 shows an XRPD pattern obtained from a sample of Form IX of endoxifen.

FIG. 11 shows an XRPD pattern obtained from a sample of Form X of endoxifen.

FIG. 12 shows an XRPD pattern obtained from a sample of Form XI of endoxifen.

FIG. 13 shows an XRPD pattern obtained from a sample of Form XII of endoxifen.

FIG. 14 shows an XRPD pattern obtained from a sample of Form XII and Form XIII of endoxifen.

FIG. 15 shows an XRPD pattern obtained from a sample of Form XIV of endoxifen.

FIG. 16 shows an XRPD pattern obtained from a sample of Form XV of endoxifen.

FIG. 17 shows an XRPD pattern obtained from a sample of Form XVII of endoxifen.

FIG. 18 shows an XRPD pattern obtained from a sample of Form XVIII of endoxifen.

FIG. 19 shows an XRPD pattern obtained from a sample of Form XIX of endoxifen.

### DETAILED DESCRIPTION

The present disclosure provides high dose oral endoxifen formulations and methods of using such formulations. Endoxifen, also referred to as 4-hydroxy-N-desmethyl-tamoxifen, is a secondary active metabolite of tamoxifen and may be used to treat a range of hormone-dependent disorders and cancers. Oral drug formulations may be easily self-administered, providing a simple and convenient means of drug delivery. However, oral delivery of endoxifen, particularly at high doses, has proved difficult due to the observed tendency of endoxifen to interconvert between the pharmaceutically active (Z)-form and the less active (E)-form under acidic conditions. Furthermore, crosslinking between endoxifen and an enteric-resistant delayed release capsule was observed in preliminary formulations of high dose endoxifen, resulting in poor dissolution and drug delivery. High dose endoxifen formulations may be advantageous for treatment regiments calling for high doses of endoxifen that may otherwise be administered as prohibitively large numbers of small dose pills.

Described herein are high dose oral endoxifen compositions that exhibit low dissolution in the acidic environment of the stomach and high dissolution in the intestinal environment. These high dose oral endoxifen compositions may be used to effectively deliver (Z)-endoxifen to a subject, providing bioavailable endoxifen in its pharmaceutically active (Z)-form. The (Z)-isoform of endoxifen may function as the more pharmaceutically active of the two isoforms. However, under acidic conditions such as in the stomach, endoxifen may readily interconvert between (Z)-endoxifen and (E)-endoxifen, as demonstrated in EXAMPLE 1, to produce an equilibrium of the two isoforms. To protect from the acidic environment of the stomach, endoxifen compositions may be encapsulated in enteric-resistant delayed release capsules, also referred to as “enteric capsules,” “enteric-resistant capsules,” “delayed-release capsules,” or “DR capsules,” that exhibit limited dissolution in the stomach and high dissolution in the intestines upon oral administration. The endoxifen compositions of the present disclosure may be formulated to prevent crosslinking between the endoxifen and the enteric-resistant delayed release capsule.

### Endoxifen Compositions

As used herein, “endoxifen” may refer to compositions comprising the (Z)-isoform, referred to as “(Z)-endoxifen,” the (E)-isoform, referred to as “(E)-endoxifen,” or a mixture of (Z)-isoform and (E)-isoform, referred to as “(E/Z)-endoxifen.” The present disclosure provides compositions, such as a high dose oral endoxifen formulation, comprising (Z)-endoxifen. In some embodiments, the (Z)-endoxifen may comprise a stable (Z)-endoxifen free base, or polymorphs or salts thereof. In some embodiments, the compositions may further comprise (E)-endoxifen. In some embodiments, the compositions may comprise endoxifen predominantly in the form of (Z)-endoxifen free base. Unless specifically referred to by the prefix (Z), (E) or (E/Z), endoxifen used generally without a prefix is used herein to include to any or all endoxifen isoforms.

A mixture of (E)-endoxifen and (Z)-endoxifen, also referred to as (E/Z)-endoxifen, can be represented by Formula (I):

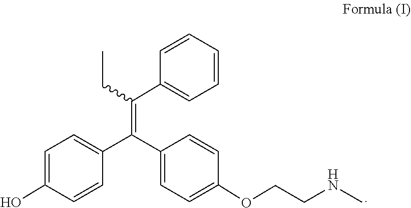 [SMILES: CCC(=C(c1ccc(O)cc1)c1ccc(OCCNC)cc1)c1ccccc1]

(Z)-endoxifen can be represented by Formula (II):

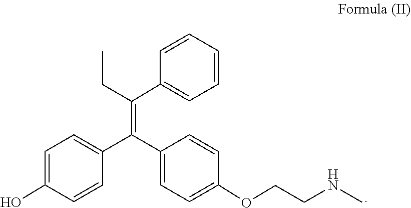 [SMILES: CC/C(=C(\c1ccc(O)cc1)c1ccc(OCCNC)cc1)c1ccccc1]

(E)-endoxifen can be represented by Formula (III):

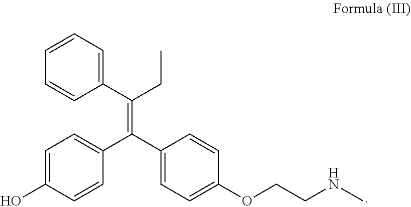 [SMILES: CC/C(=C(/c1ccc(O)cc1)c1ccc(OCCNC)cc1)c1ccccc1]

In some embodiments, the endoxifen compositions of the present disclosure may comprise (Z)-endoxifen as at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.5%, at least about 99.99%, or about 100% of total endoxifen (e.g., (Z)-endoxifen and (E)-endoxifen), by weight, in the composition. In some embodiments, the composition comprises at least 90% of (Z)-endoxifen with respect to total endoxifen, by weight, in the composition. In some embodiments, the composition comprises at least 95% of (Z)-endoxifen with respect to total endoxifen, by weight, in the composition. In some embodiments, the composition comprises at least about 96%, at least about 97%, at least about 98%, at least about 99%, at least about 99.5%, or at least about 99.9% of (Z)-endoxifen with respect to total endoxifen, by weight, in the composition. In some embodiments, the endoxifen compositions of the present disclosure may comprise (E)-endoxifen as no more than about 10%, no more than about 8%, no more than about 6%, no more than about 7%, no more than about 8%, no more than about 9%, no more than about 2%, or no more than about 1% of total endoxifen (e.g., (Z)-endoxifen and (E)-endoxifen), by weight, in the composition.

In some embodiments, the endoxifen compositions of the present disclosure may comprise a ratio of (Z)-endoxifen to (E)-endoxifen (Z:E ratio) that is at least about 50:50, at least about 60:40, at least about 64:36, at least about 70:30, at least about 80:30, at least about 82:18, at least about 85:15, at least about 90:10, at least about 94:6, at least about 95:5, at least about 96:4, at least about 97:3, at least about 98:2, at least about 99:1, or about 100:0. In some embodiments, the endoxifen compositions of the present disclosure may comprise a ratio of (Z)-endoxifen to (E)-endoxifen (Z:E ratio) that is from about 50:50 to about 100:0, from about 60:40 to about 100:0, from about 64:36 to about 100:0, from about 70:30 to about 100:0, from about 80:30 to about 100:0, from about 82:18 to about 100:0, from about 85:15 to about 100:0, from about 90:10 to about 100:0, from about 94:6 to about 100:0, from about 95:5 to about 100:0, from about 96:4 to about 100:0, from about 97:3 to about 100:0, from about 98:2 to about 100:0, or from about 99:1 to about 100:0.

A composition comprising (Z)-endoxifen may comprise at least about 4% (Z)-endoxifen, by weight, relative to total fill weight of the composition (e.g., excluding, if present, a capsule or coating encapsulating the composition). A composition comprising (Z)-endoxifen may comprise at least about 10% (Z)-endoxifen, by weight, relative to total fill weight of the composition. In some embodiments, the composition comprising (Z)-endoxifen may comprise at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, or at least about 20% (Z)-endoxifen, by weight, relative to total fill weight of the composition. In some embodiments, the composition comprising (Z)-endoxifen may comprise from about 4% to about 50%, from about 5% to about 50%, from about 7% to about 50%, from about 10% to about 50%, from about 12% to about 50%, from about 15% to about 50%, from about 4% to about 40%, from about 5% to about 40%, from about 7% to about 40%, from about 10% to about 40%, from about 12% to about 40%, from about 15% to about 40%, from about 4% to about 30%, from about 5% to about 30%, from about 7% to about 30%, from about 10% to about 30%, from about 12% to about 30%, from about 15% to about 30%, from about 4% to about 20%, from about 5% to about 20%, from about 7% to about 20%, from about 10% to about 20%, from about 12% to about 20%, or from about 15% to about 20% (Z)-endoxifen, by weight, relative to total fill weight of the composition.

A composition of the present disclosure (e.g., an enteric-resistant delayed release high dose endoxifen formulation) may comprise an amount of (Z)-endoxifen formulated as a dosage form, such as a tablet or capsule. In some embodiments, a single high dose endoxifen dosage form (e.g., a single capsule or a single tablet) may comprise about 10 mg, about 12 mg, about 15 mg, about 16 mg, about 20 mg, about 22 mg, about 24 mg, about 25 mg, about 28 mg, about 30 mg, about 32 mg, about 35 mg, about 36 mg, about 38 mg, about 40 mg, about 42 mg, about 45 mg, about 50 mg, about 60 mg, about 70 mg, about 80 mg, about 90 mg, or about 100 mg of (Z)-endoxifen. In some embodiments, a single high dose endoxifen dosage form may comprise from about 10 mg to about 100 mg, from about 10 mg to about 80 mg, from about 10 mg to about 50 mg, from about 20 mg to about 50 mg, from about 30 mg to about 50 mg, or from about 10 mg to about 30 mg of (Z)-endoxifen.

Endoxifen, also referred to as 4-hydroxy-N-desmethyl-tamoxifen, may include a polymorphic, salt, free base, co-crystal, or solvate form of endoxifen. Examples of salts of (Z)-endoxifen suitable for the endoxifen compositions of the present disclosure (e.g., enteric-resistant delayed release high dose endoxifen formulations) include pharmacologically acceptable salts such as salts with inorganic acids, salts with organic acids, salts with amino acids and the like. Examples of (Z)-endoxifen salts with inorganic acids include salts with hydrochloric acid, hydrobromic acid, nitric acid, sulfuric acid, phosphoric acid, and the like. Examples of salts with organic acids include salts with formic acid, acetic acid, trifluoroacetic acid, fumaric acid, oxalic acid, tartaric acid, maleic acid, methanesulfonic acid, benzenesulfonic acid, p-toluenesulfonic acid, and the like. Examples of the salts with acidic amino acids include salts with aspartic acid, glutamic acid, citric acid, and the like.

Examples of anion salts of (Z)-endoxifen include arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, and triethiodide. Examples of cation salts of (Z)-endoxifen selected from the group consisting of benzathine, clemizole, chloroprocaine, choline, diethylamine, diethanolamine, ethylenediamine, meglumine, piperazine, procaine, aluminum, barium, bismuth, lithium, magnesium, potassium, and zinc, and the like. In some embodiments, the present disclosure provides that embodiments include salts made with acids that are not pharmaceutically acceptable.

In some embodiments, an endoxifen composition of the present disclosure comprises salts of (Z)-endoxifen selected from the group consisting of acetate, arecoline, benzathine, benzoic, besylate, benzosulfonate, bicarbonate, bitartarate, butylbromide, citrate, camysylate, clemizole, chloroprocaine, choline, diethylamine, diethanolamine, ethylenediamine, formate, fumarate, glucolate, gluconate, glutamate, glycollylarsanilate, hexylresorcinate, hydrabamine, hydrobromide, hydrochloride, hydroxynapthanoate, isethionate, malate, maleate, mandelate, meglumine, mesylate, methylbromide, methylbromide, methylnitrate, methylsulfate, methanesulfonate, mucate, napsylate, nitric, nitrate, oxalate, pamaoate (Embonate), pantothenate, perchloric, phosphate, diphosphate, piperazine, procaine, polygalacuronate, p-toluenesulfonate, salicylate, stearate, succinate, sulfate, sulfonate, sulfuric, tannate, tartarate, teoclate, triethiodide, trifluoroacetate, aluminum, barium, bismuth, lithium, magnesium, potassium, and zinc aluminum, barium, bismuth, lithium, magnesium, potassium, and zinc, or any combination thereof. In some embodiments, the salt is (Z)-endoxifen gluconate. Endoxifen gluconate can be selected from the group consisting of (Z)-endoxifen D-gluconate, (Z)-endoxifen L-gluconate, or a combination thereof.

While the content of (Z)-endoxifen or a polymorph or a salt thereof in the endoxifen compositions of the present disclosure varies depending on the dosage form of the composition, target disease, severity of disease, and the like, it is an amount generally corresponding or equivalent to from about 0.01 mg to about 200 mg of (Z)-endoxifen. One of skill in the art will recognize that when a composition includes salts of (Z)-endoxifen, the endoxifen salt may be in an equivalent amount on the basis of (Z)-endoxifen to be released.

In some embodiments, the endoxifen may comprise one or more polymorphic forms, such as Form I, of endoxifen. A polymorphic form may be distinguished by its x-ray powder diffraction pattern. In some embodiments, a method of treating a cancer may comprise administering a pharmaceutical composition comprising endoxifen predominantly as polymorph Form I. In some embodiments, polymorphic Form I is characterized by an x-ray powder diffraction pattern comprising major peaks at 16.8±0.3°, 17.1±0.3° and 21.8±0.3° two theta.

The endoxifen in a composition of the present disclosure may be present in one or more polymorphic forms (e.g., Form I). For example, a composition of the present disclosure may comprise at least about 90% of total endoxifen, by weight, as polymorph Form I. In another example, a composition of the present disclosure may comprise at least about 95% of total endoxifen, by weight, as polymorph Form I. When a particular percentage by weight of endoxifen is a single polymorphic form (e.g., Form I), the remainder of endoxifen in the composition may be some combination of amorphous endoxifen or one or more polymorphic forms of endoxifen excluding the single polymorphic form. In some embodiments, the endoxifen composition comprises (Z)-endoxifen predominantly as polymorph Form I.

In certain aspects, the present disclosure provides crystalline forms of endoxifen, including crystalline forms of (Z)-endoxifen free base and crystalline forms of mixtures of (E)-endoxifen and (Z)-endoxifen. The present disclosure further provides pharmaceutical compositions of endoxifen comprising the crystalline forms described herein. A crystalline form of endoxifen may provide the advantage of bioavailability and stability, suitable for use as an active ingredient in a pharmaceutical composition. Variations in the crystal structure of a pharmaceutical drug substance or active ingredient may affect the dissolution rate (which may affect bioavailability, etc.), manufacturability (such as ease of handling, ability to consistently prepare doses of known strength) and stability (such as thermal stability, shelf life, etc.) of a pharmaceutical drug product or active ingredient. Such variations may affect the preparation or formulation of pharmaceutical compositions in different dosage or delivery forms, such as solid oral dosage forms including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, process control, or combinations thereof. Thus, crystalline forms of endoxifen may provide advantages such as: improving the manufacturing process of an active agent or the stability or storability of a drug product form of the compound or an active ingredient, and/or having suitable bioavailability and/or stability as an active agent.

The use of certain solvents and fractional crystallization methods may produce different polymorphic forms of endoxifen which may exhibit one or more favorable characteristics described above. In some embodiments, a polymorphic form (e.g., Form I) may affect one or more properties of a composition comprising endoxifen. For example, a polymorphic form (e.g., Form I) of a therapeutic agent (e.g., endoxifen) may affect one or more of the dissolution rate, the solubility, the absorption rate, the Cₘₐₓ, the AUC, the Tₘₐₓ, or the t_{1/2}, of the therapeutic agent in a composition of the present disclosure. In some embodiments, the polymorphic form of endoxifen may confer one or more properties that favorably contribute to manufacturability of a composition of the present disclosure (e.g., an enteric-resistant delayed release high dose endoxifen formulation). In some embodiments, the polymorphic form of endoxifen may confer improved stability to a composition of the present disclosure. The processes for the preparation of the polymorphs described herein, and characterization of these polymorphs are described in greater detail below.

### Polymorphic Forms of Endoxifen

A composition of the present disclosure may comprise a polymorphic form of endoxifen (e.g., (Z)-endoxifen). In some embodiments, the endoxifen may comprise one or more polymorphic forms, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX of endoxifen. In some embodiments, a polymorphic form may be a crystalline form. A polymorphic form may be distinguished by its x-ray powder diffraction pattern. In some embodiments, the composition may comprise endoxifen predominantly as polymorph Form I. In some embodiments, the composition may comprise endoxifen predominantly as polymorph Form II. In some embodiments, the composition may comprise endoxifen predominantly as polymorph Form III. In some embodiments, the composition may comprise endoxifen predominantly as polymorph Form IV. In some embodiments, the composition may comprise endoxifen predominantly as polymorph Form V. In some embodiments, the composition may comprise endoxifen predominantly as polymorph Form VI. In some embodiments, the composition may comprise endoxifen predominantly as polymorph Form VII. In some embodiments, the composition may comprise endoxifen predominantly as polymorph Form VIII. In some embodiments, the composition may comprise endoxifen predominantly as polymorph Form IX. In some embodiments, the composition may comprise endoxifen predominantly as polymorph Form X. In some embodiments, the composition may comprise endoxifen predominantly as polymorph Form XI. In some embodiments, the composition may comprise endoxifen predominantly as polymorph Form XII. In some embodiments, the composition may comprise endoxifen predominantly as polymorph Form XIII. In some embodiments, the composition may comprise endoxifen predominantly as polymorph Form XIV. In some embodiments, the composition may comprise endoxifen predominantly as polymorph Form XV. In some embodiments, the composition may comprise endoxifen predominantly as polymorph Form XVII. In some embodiments, the composition may comprise endoxifen predominantly as polymorph Form XVIII. In some embodiments, the composition may comprise endoxifen predominantly as polymorph Form XIX.

In some embodiments, at least 90% of the endoxifen in the composition may be polymorphic Form I. In some embodiments, at least 90% of the endoxifen in the composition may be polymorphic Form II. In some embodiments, at least 90% of the endoxifen in the composition may be polymorphic Form III. In some embodiments, at least 90% of the endoxifen in the composition may be polymorphic Form IV. In some embodiments, at least 90% of the endoxifen in the composition may be polymorphic Form V. In some embodiments, at least 90% of the endoxifen in the composition may be polymorphic Form VI. In some embodiments, at least 90% of the endoxifen in the composition may be polymorphic Form VII. In some embodiments, at least 90% of the endoxifen in the composition may be polymorphic Form VIII. In some embodiments, at least 90% of the endoxifen in the composition may be polymorphic Form IX. In some embodiments, at least 90% of the endoxifen in the composition may be polymorphic Form X. In some embodiments, at least 90% of the endoxifen in the composition may be polymorphic Form XI. In some embodiments, at least 90% of the endoxifen in the composition may be polymorphic Form XII. In some embodiments, at least 90% of the endoxifen in the composition may be polymorphic Form XII. In some embodiments, at least 90% of the endoxifen in the composition may be polymorphic Form XIV. In some embodiments, at least 90% of the endoxifen in the composition may be polymorphic Form XV. In some embodiments, at least 90% of the endoxifen in the composition may be polymorphic Form XVII. In some embodiments, at least 90% of the endoxifen in the composition may be polymorphic Form XVIII. In some embodiments, at least 90% of the endoxifen in the composition may be polymorphic Form XIX.

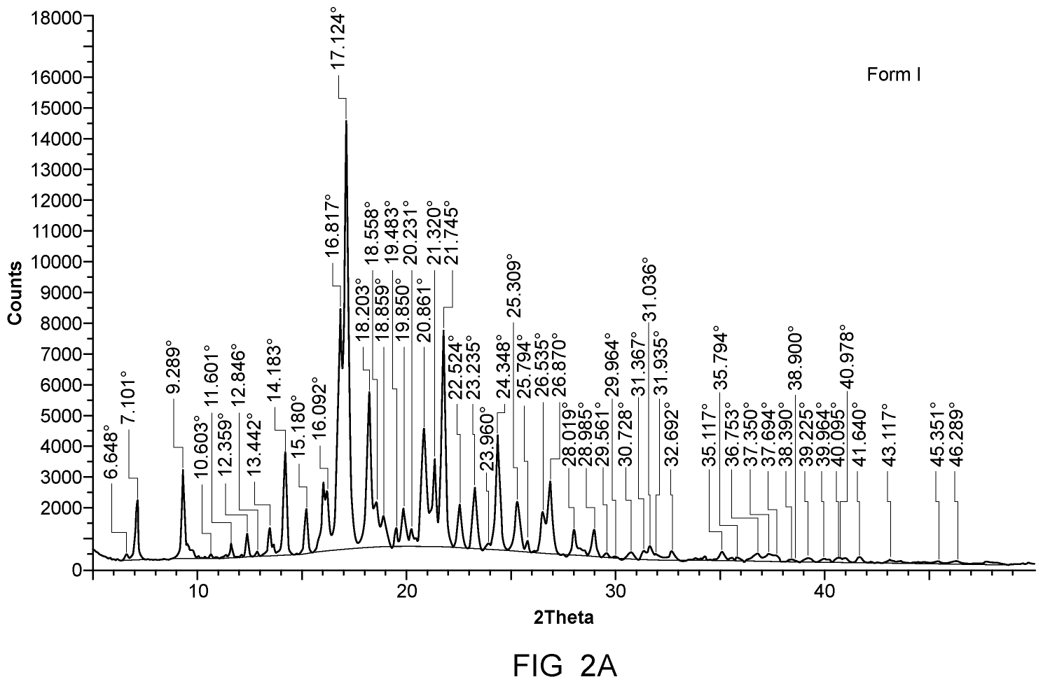

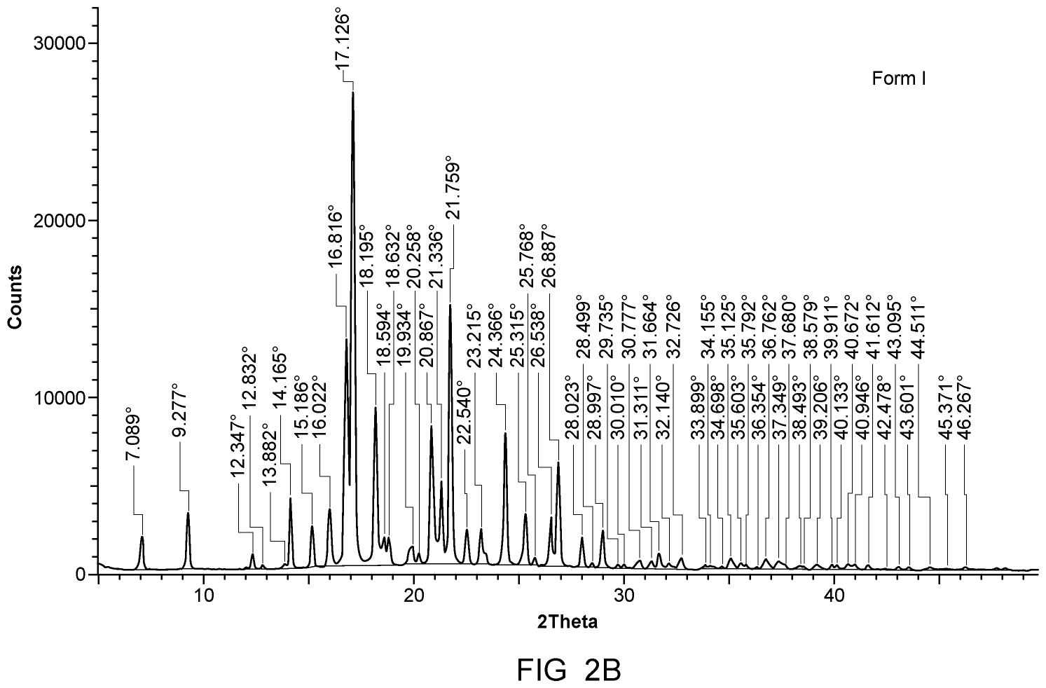

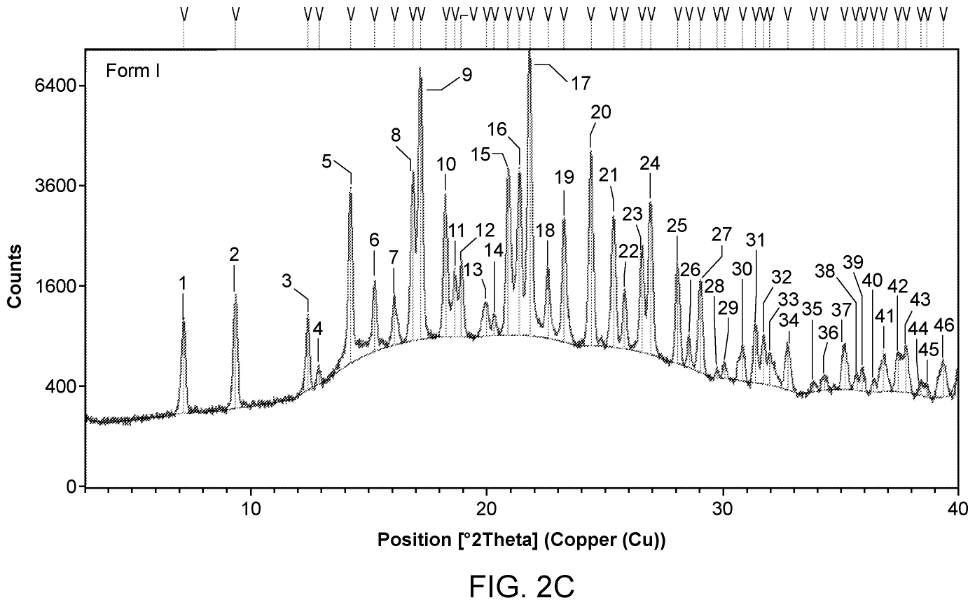

In certain aspects, the present disclosure provides compositions comprising polymorphic Form I of endoxifen. In some embodiments, at least 90% by weight of the endoxifen in the composition is the (Z)-isomer (i.e., (Z)-endoxifen). In some embodiments, polymorphic Form I of endoxifen exhibits an x-ray powder diffraction (XRPD) pattern substantially as shown in FIG. 2A, FIG. 2B, or FIG. 2C. In some embodiments, polymorphic Form I has an XRPD pattern comprising at least two, at least three, at least four, at least five, or at least six of the major peaks as the XRPD pattern substantially as shown in FIG. 2A, FIG. 2B, or FIG. 2C. The crystalline form of Form I of the compound of Formula (III) can be characterized by an XRPD pattern comprising major peaks at 16.8±0.3°, 17.1±0.3° and 21.8±0.3° two theta. In some cases, the XRPD pattern further comprises at least one peak, or at least two peaks, at least three peaks selected from 16.0±0.3°, 18.8±0.3° and 26.5±0.3° two theta. In some cases, the XRPD pattern further comprises at least one peak, or at least two peaks, at least three peaks selected from 12.3±0.3°, 28.0±0.3° and 29.0±0.3° two theta.

The term “substantially as shown in” when referring, for example, to an XRPD pattern, includes a pattern that is not necessarily identical to those depicted herein, but that falls within the limits of experimental error or deviations when considered by one of ordinary skill in the art. The relative intensities of XRPD peaks can vary, depending upon the particle size, the sample preparation technique, the sample mounting procedure, and the particular instrument employed. Moreover, instrument variation and other factors can affect the two theta (20) values. Accordingly, when a specified two theta angle is provided, it is to be understood that the specified two theta angle can vary by the specified value±0.5°, such as ±0.4°, ±0.3°, ±0.2°, or ±0.1°. As used herein, “major peak” refers to an XRPD peak with a relative intensity greater than 30%, such as greater than 35%. Relative intensity is calculated as a ratio of the peak intensity of the peak of interest versus the peak intensity of the largest peak in the XRPD pattern.

Polymorphic Form I may be characterized by an x-ray powder diffraction pattern comprising major peaks at 16.8±0.3°, 17.1±0.3° and 21.8±0.3° two theta. In some embodiments, the x-ray powder diffraction pattern of polymorphic Form I may be further characterized by one or more peaks selected from 16.0±0.3°, 18.8±0.3° and 26.5±0.3° two theta. In some embodiments, the x-ray powder diffraction pattern of polymorphic Form I may be further characterized by one or more peaks selected from 12.3±0.3°, 28.0±0.3° and 29.0±0.3° two theta. In some embodiments, the x-ray powder diffraction pattern of polymorphic Form I may be further characterized by one or more peaks selected from 12.3±0.3°, 16.0±0.3°, 18.8±0.3°, 26.5±0.3°, 28.0±0.3° and 29.0±0.3° two theta.

In some embodiments, the present disclosure provides a composition comprising polymorphic Form I of endoxifen. Greater than 90%, 95% or 99% by weight of the endoxifen in the composition may be polymorphic Form I. In some embodiments, the composition comprises 0.01 mg to 200 mg of polymorphic Form I. In some embodiments, at least about 5%, at least about 10%, about at least 20%, at least about 25%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, at least about 99.5%, at least about 99.99%, or about 100% of total endoxifen, by weight, in a composition may be present as polymorphic Form I.

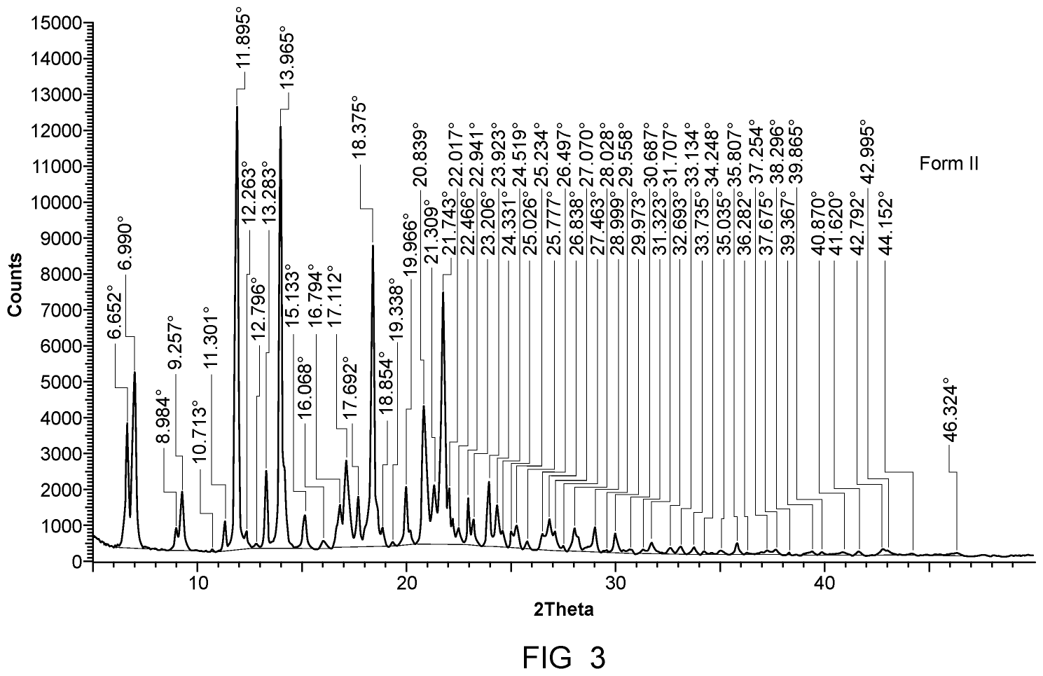

In some embodiments, polymorphic Form II is characterized by an x-ray powder diffraction pattern shown in FIG. 3. The crystalline form of Form II of the compound of Formula (III) can be characterized by an XRPD pattern comprising major peaks at 7.0±0.3°, 11.9±0.3°, 14.0±0.3° and 18.4±0.3° two theta. The crystalline form of Form II of the compound of Formula (III) may be characterized by an x-ray powder diffraction pattern comprising major peaks at 7.0±0.3°, 11.9±0.3°, and 14.0±0.3° two theta. In some cases, the XRPD pattern further comprises at least one peak, or at least two peaks, at least three peaks, or at least four peaks selected from 18.4±0.3°, 22.0±0.3°, 6.6±0.3°, and 13.3±0.3° two theta. In some cases, the XRPD pattern further comprises at least one peak, or at least two peaks, at least three peaks, at least four peaks, or at least five peaks selected from 20.0±0.3°, 6.6±0.3°, 13.3±0.3°, 20.0±0.3° and 22.0±0.3° two theta.

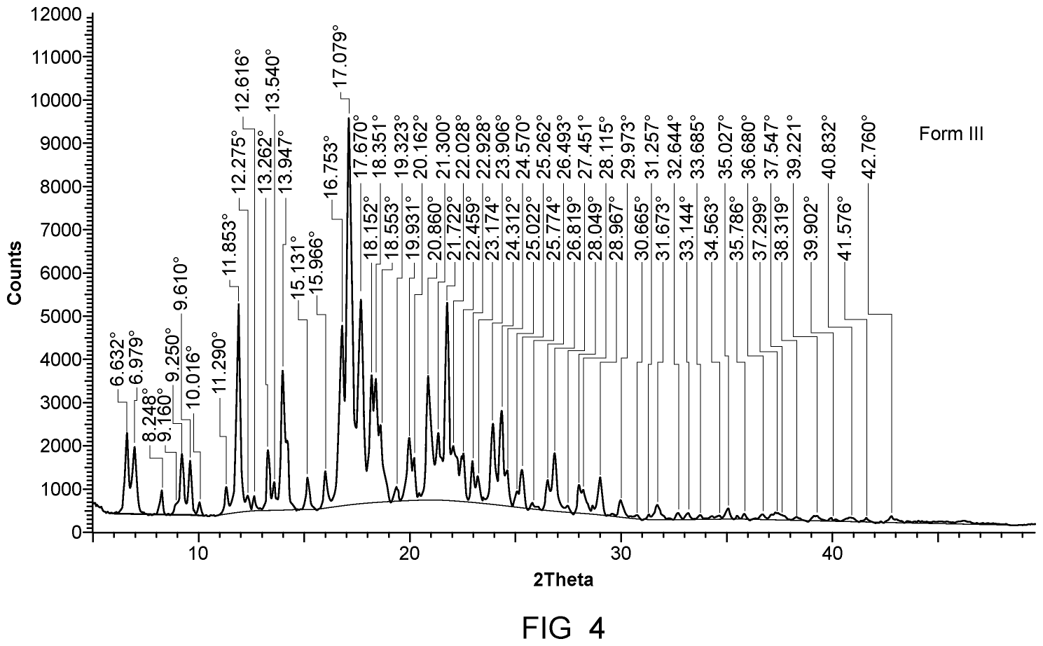

In some embodiments, polymorphic Form III is characterized by an x-ray powder diffraction pattern, shown in FIG. 4. The crystalline form of Form III of the compound of Formula (III) can be characterized by an XRPD pattern comprising major peaks at 11.9±0.3°, 13.9±0.3°, and 17.1±0.3° two theta. In some cases, the XRPD pattern further comprises at least one peak, or at least two peaks, at least three peaks, or at least four peaks selected from 17.7±0.3°, 25.3±0.3°, 18.2±0.3°, and 22.5±0.3° two theta. In some cases, the XRPD pattern further comprises at least one peak, or at least two peaks, at least three peaks, at least four peaks, or at least five peaks selected from 26.8±0.3°, 18.2±0.3°, 22.5±0.3°, 25.3±0.3° and 26.8±0.3° two theta.

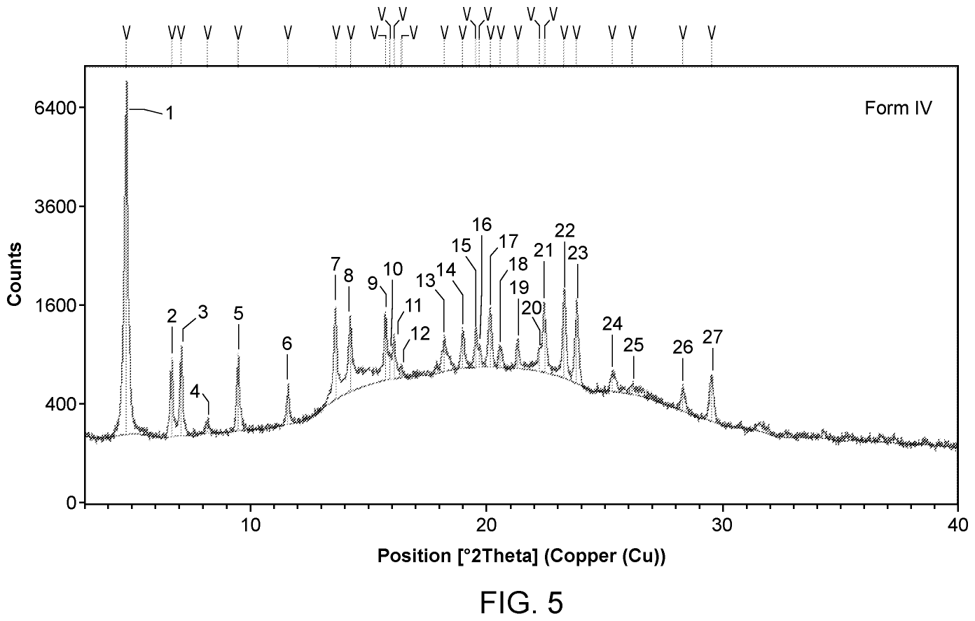

In some embodiments, polymorphic Form IV may be characterized by an x-ray powder diffraction pattern shown in FIG. 5. The crystalline form of Form IV of the compound of Formula (III) can be characterized by an XRPD pattern comprising major peaks at 4.7±0.3° two theta, 23.3±0.3°, and 13.6±0.3° two theta. In some cases, the XRPD pattern further comprises at least one peak, at least two peaks, at least three peaks, or at least four peaks selected from 23.8±0.3°, 14.2±0.3°, 22.5±0.3°, or 15.7±0.3° two theta. In some cases, the XRPD pattern further comprises at least one peak, at least two peaks, or at least three peaks selected from 7.1±0.3°, 20.2±0.3°, or 9.5±0.3° two theta. In some cases, the XRPD pattern further comprises at least one peak, comprises at least two peaks, at least three peaks, at least four peaks, at least five peaks, or at least six peaks selected from 7.1±0.3°, 9.5±0.3°, 14.2±0.3°, 15.7±0.3°, 20.2±0.3°, 22.5±0.3°, and 23.8±0.3° two theta.

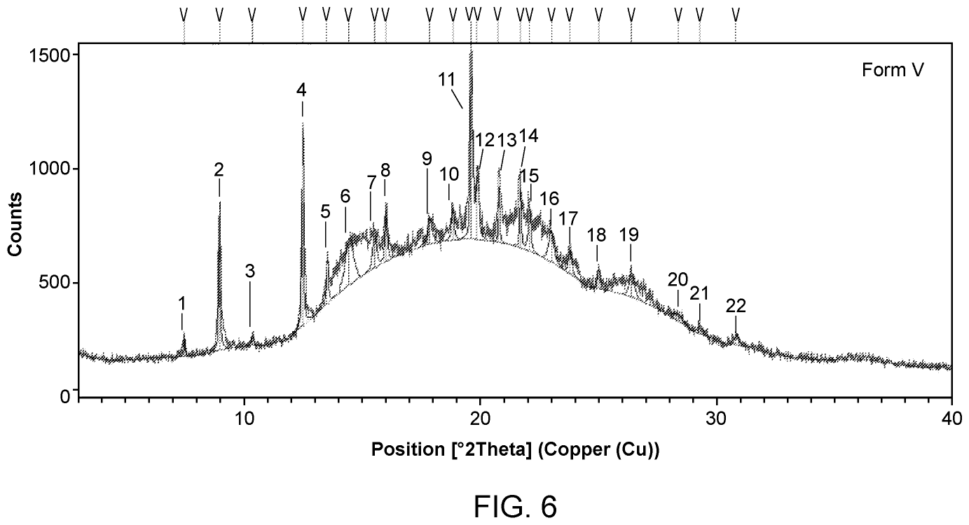

In some embodiments, polymorphic Form V may be characterized by an x-ray powder diffraction pattern shown in FIG. 6. The crystalline form of Form V of the compound of Formula (III) can be characterized by an XRPD pattern comprising major peaks at 12.5±0.3°, 19.6±0.3°, and 8.9±0.3° two theta. In some cases, the XRPD pattern further comprises at least one peak, at least two peaks, at least three peaks, or at least four peaks selected from 21.7±0.3°, 20.8±0.3°, 19.8±0.3°, or 16.0±0.3° two theta. In some cases, the XRPD pattern further comprises at least one peak, at least two peaks, or at least three peaks selected from 22.0±0.3°, 13.5±0.3°, or 14.4±0.3° two theta. In some cases, the XRPD pattern further comprises at least one peak, comprises at least two peaks, at least three peaks, at least four peaks, at least five peaks, or at least six peaks selected from 21.7±0.3°, 20.8±0.3°, 19.8±0.3°, 16.0±0.3°, 22.0±0.3°, 13.5±0.3°, and 14.4±0.3° two theta.

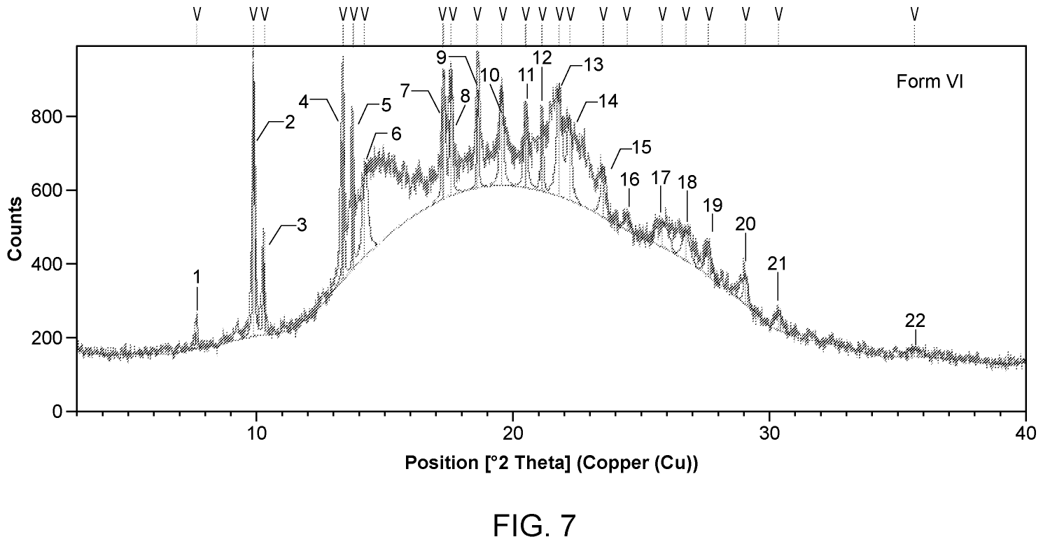

In some embodiments, polymorphic Form VI may be characterized by an x-ray powder diffraction pattern shown in FIG. 7. The crystalline form of Form VI of the compound of Formula (III) can be characterized by an XRPD pattern comprising major peaks at 9.9±0.3°, 13.4±0.3°, and 13.7±0.3° two theta. In some cases, the XRPD pattern further comprises at least one peak, at least two peaks, at least three peaks, or at least four peaks selected from 17.6±0.3°, 18.6±0.3°, 17.3±0.3°, or 21.8±0.3° two theta. In some cases, the XRPD pattern further comprises at least one peak, at least two peaks, or at least three peaks selected from 10.2±0.3°, 19.5±0.3°, or 14.2±0.3° two theta. In some cases, the XRPD pattern further comprises at least one peak, comprises at least two peaks, at least three peaks, at least four peaks, at least five peaks, or at least six peaks selected from 17.6±0.3°, 18.6±0.3°, 17.3±0.3°, 21.8±0.3°, 10.2±0.3°, 19.5±0.3°, or 14.2±0.3° two theta.

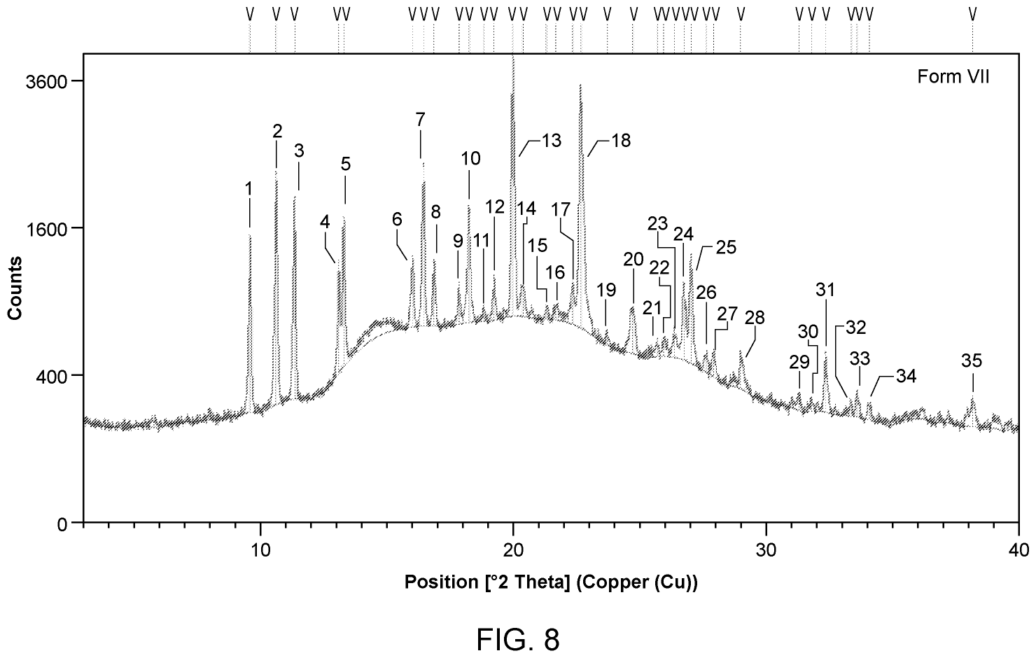

In some embodiments, polymorphic Form VII may be characterized by an x-ray powder diffraction pattern shown in FIG. 8. The crystalline form of Form VII of the compound of Formula (III) can be characterized by an XRPD pattern comprising major peaks at 20.0±0.3°, 22.6±0.3°, and 10.6±0.3° two theta. In some cases, the XRPD pattern further comprises at least one peak, at least two peaks, at least three peaks, or at least four peaks selected from 11.4±0.3°, 16.4±0.3°, 9.6±0.3°, or 13.3±0.3° two theta. In some cases, the XRPD pattern further comprises at least one peak, at least two peaks, or at least three peaks selected from 18.2±0.3°, 13.1±0.3°, or 27.0±0.3° two theta. In some cases, the XRPD pattern further comprises at least one peak, comprises at least two peaks, at least three peaks, at least four peaks, at least five peaks, or at least six peaks selected from 11.4±0.3°, 16.4±0.3°, 9.6±0.3°, 13.3±0.3°, 18.2±0.3°, 13.1±0.3°, or 27.0±0.3° two theta.

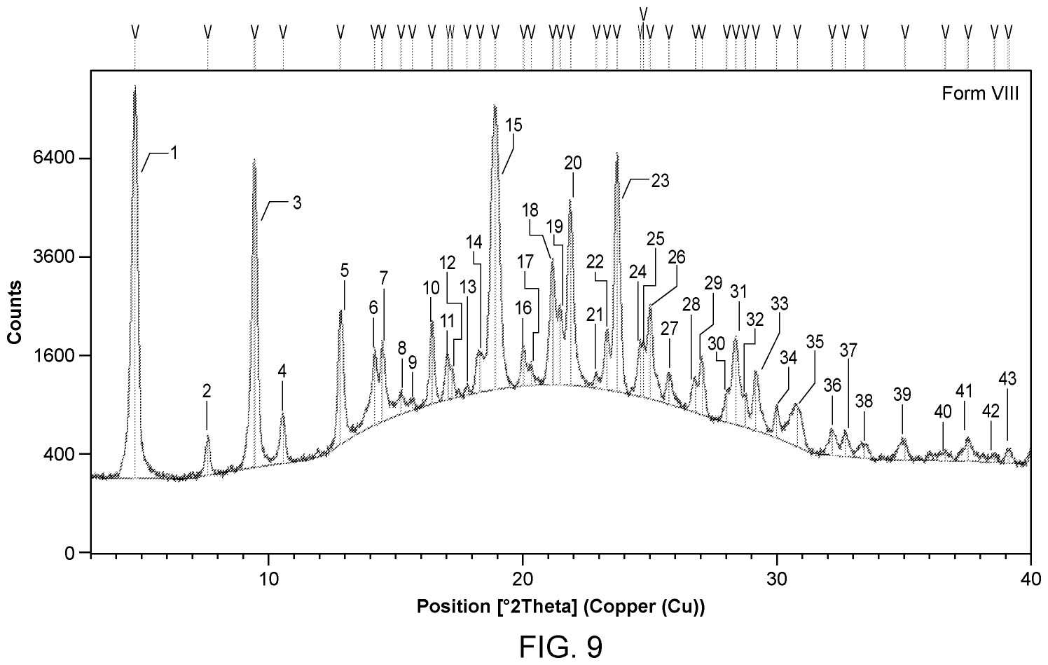

In some embodiments, polymorphic Form VIII may be characterized by an x-ray powder diffraction pattern shown in FIG. 9. The crystalline form of Form VIII of the compound of Formula (III) can be characterized by an XRPD pattern comprising major peaks at 4.8±0.3°, 18.9±0.3°, and 9.5±0.3° two theta. In some cases, the XRPD pattern further comprises at least one peak, at least two peaks, at least three peaks, or at least four peaks selected from 23.7±0.3°, 21.9±0.3°, 21.2±0.3°, or 12.9±0.3° two theta. In some cases, the XRPD pattern further comprises at least one peak, at least two peaks, or at least three peaks selected from 25.0±0.3°, 21.5±0.3°, or 16.4±0.3° two theta. In some cases, the XRPD pattern further comprises at least one peak, comprises at least two peaks, at least three peaks, at least four peaks, at least five peaks, or at least six peaks selected from 23.7±0.3°, 21.9±0.3°, 21.2±0.3°, 12.9±0.3°, 25.0±0.3°, 21.5±0.3°, or 16.4±0.3° two theta.

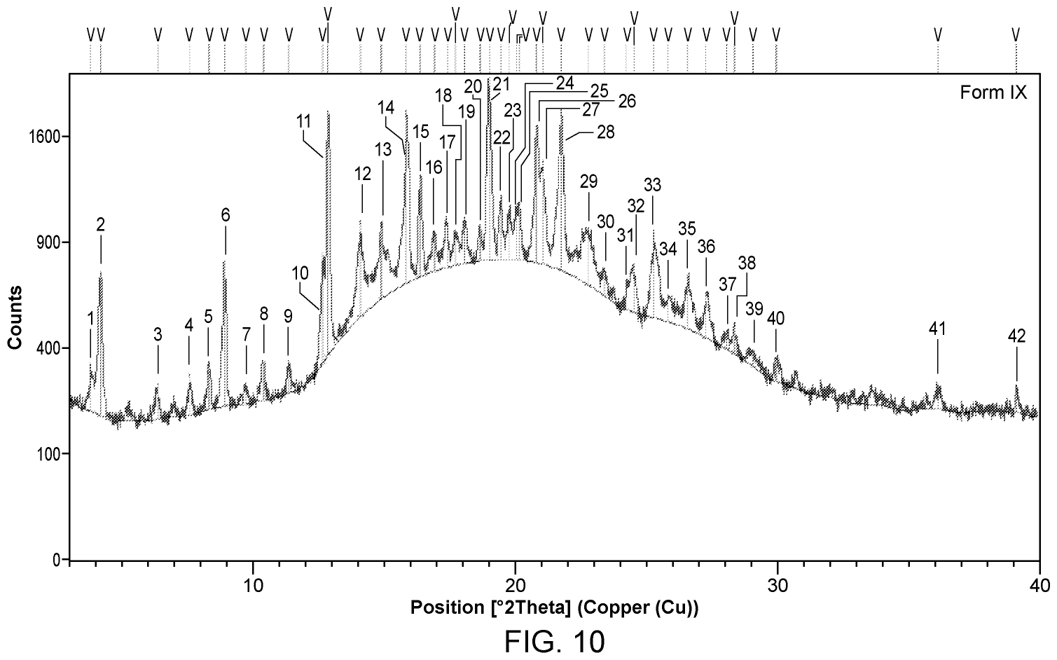

In some embodiments, polymorphic Form IX may be characterized by an x-ray powder diffraction pattern shown in FIG. 10. The crystalline form of Form IX of the compound of Formula (III) can be characterized by an XRPD pattern comprising major peaks at 19.0±0.3°, 12.9±0.3°, and 15.9±0.3° two theta. In some cases, the XRPD pattern further comprises at least one peak, at least two peaks, at least three peaks, or at least four peaks selected from 21.7±0.3°, 20.8±0.3°, 21.1±0.3°, or 8.9±0.3° two theta. In some cases, the XRPD pattern further comprises at least one peak, at least two peaks, or at least three peaks selected from 16.4±0.3°, 4.2±0.3°, or 12.7±0.3° two theta. In some cases, the XRPD pattern further comprises at least one peak, comprises at least two peaks, at least three peaks, at least four peaks, at least five peaks, or at least six peaks selected from 21.7±0.3°, 20.8±0.3°, 21.1±0.3°, 8.9±0.3°, 16.4±0.3°, 4.2±0.3°, or 12.7±0.3° two theta.

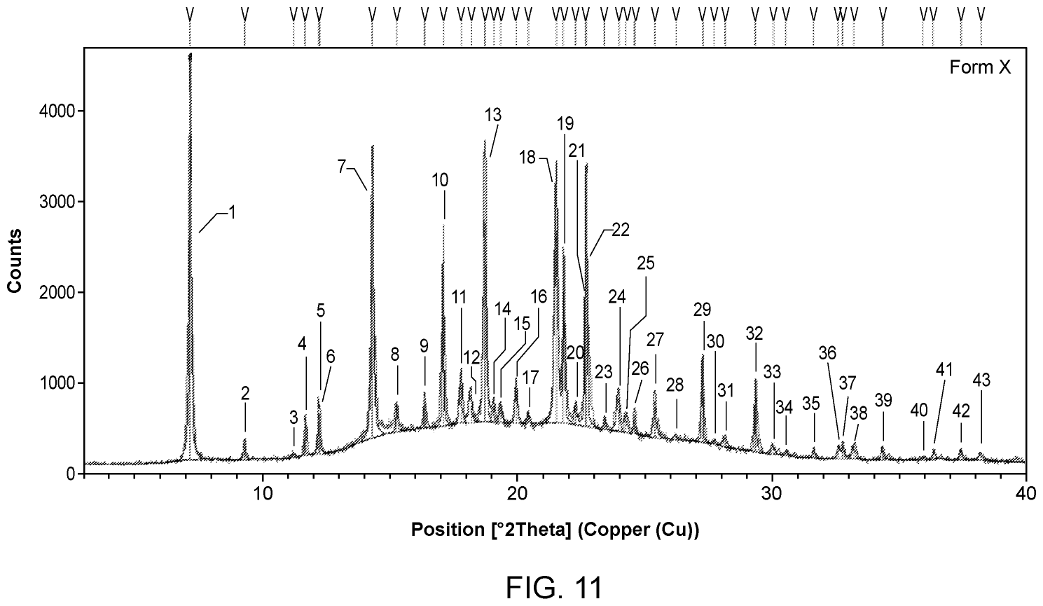

In some embodiments, polymorphic Form X may be characterized by an x-ray powder diffraction pattern shown in FIG. 11. The crystalline form of Form X of the compound of Formula (III) can be characterized by an XRPD pattern comprising major peaks at 7.2±0.3°, 14.3±0.3°, and 18.7±0.3° two theta. In some cases, the XRPD pattern further comprises at least one peak, at least two peaks, or at least three peaks selected from 21.5±0.3°, and 22.7±0.3°, and 17.1±0.3° two theta. In some cases, the XRPD pattern further comprises at least one peak, at least two peaks, or at least three peaks selected from 21.8±0.3°, 27.3±0.3°, or 29.4±0.3° two theta.

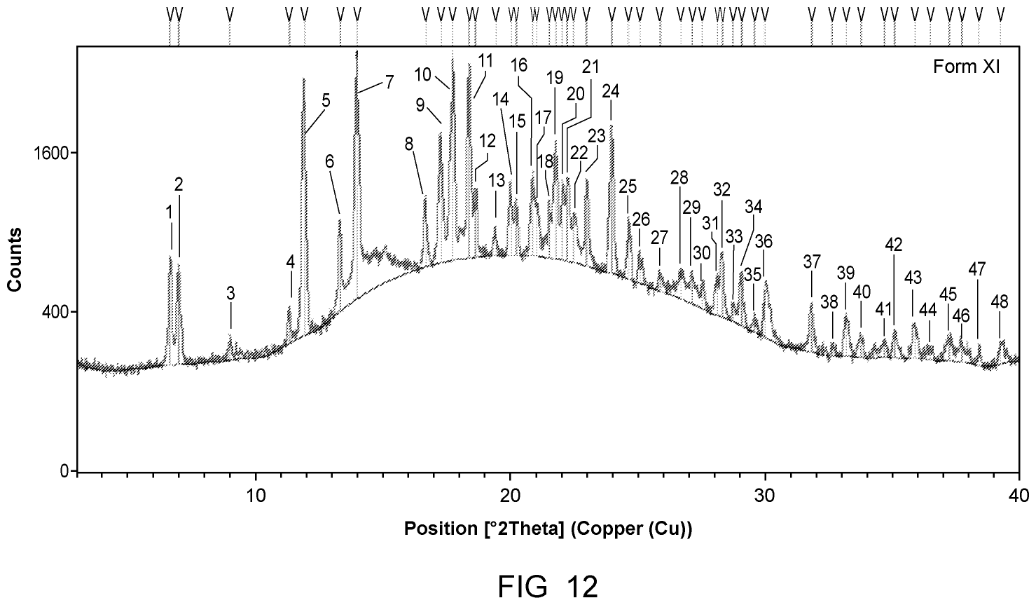

In some embodiments, polymorphic Form XI may be characterized by an x-ray powder diffraction pattern shown in FIG. 12. The crystalline form of Form XI of the compound of Formula (III) can be characterized by an XRPD pattern comprising major peaks at 14.0±0.3°, 17.7±0.3°, and 11.9±0.3° two theta. In some cases, the XRPD pattern further comprises at least one peak, at least two peaks, or at least three peaks selected from 18.4±0.3°, 23.9±0.3°, or 17.3±0.3° two theta. In some cases, the XRPD pattern further comprises at least one peak, at least two peaks, at least three peaks, or at least four peaks selected from 21.8±0.3°, 20.8±0.3°, and 23.0±0.3°, or 22.2±0.3° two theta.

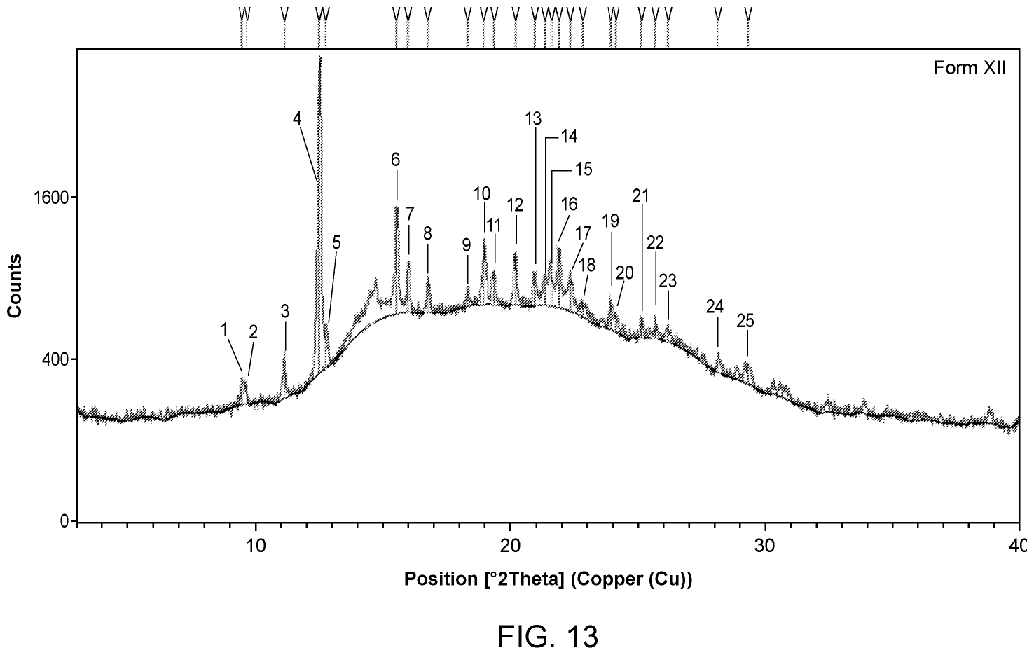

In some embodiments, polymorphic Form XII may be characterized by an x-ray powder diffraction pattern shown in FIG. 13. The crystalline form of Form XII of the compound of Formula (III) can be characterized by an XRPD pattern comprising major peaks at 12.5±0.3°, 15.6±0.3°, and 19.0±0.3° two theta. In some cases, the XRPD pattern further comprises at least one peak, at least two peaks, at least three peaks, or at least four peaks selected from 21.9±0.3°, 20.2±0.3°, 16.0±0.3°, or 21.6±0.3° two theta. In some cases, the XRPD pattern further comprises at least one peak, at least two peaks, or at least three peaks selected from 22.4±0.3°, 16.8±0.3°, or 12.8±0.3° two theta.

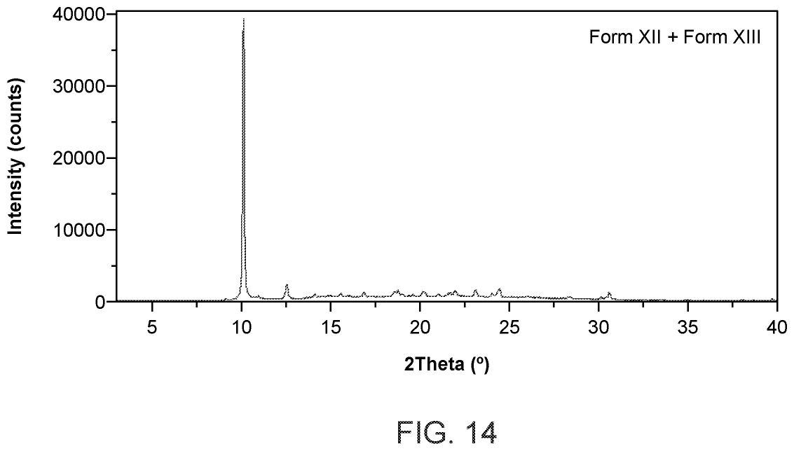

In some embodiments, polymorphic Form XIII in a mixture with Form XII may be characterized by an x-ray powder diffraction pattern shown in FIG. 14.

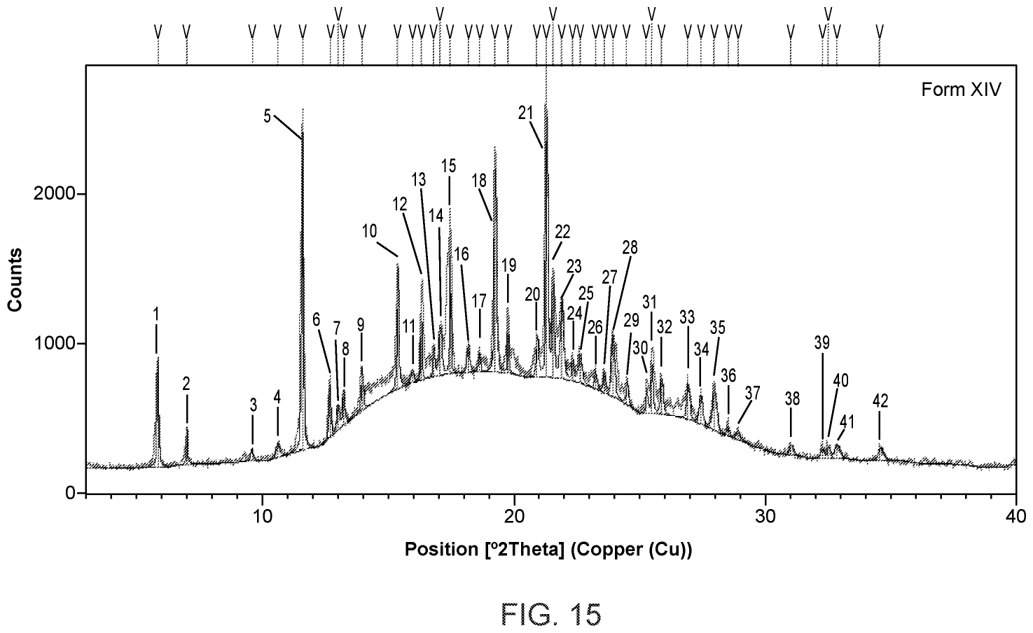

In some embodiments, polymorphic Form XIV may be characterized by an x-ray powder diffraction pattern shown in FIG. 15. The crystalline form of Form XIV of the compound of Formula (III) can be characterized by an XRPD pattern comprising major peaks at 11.6±0.3°, 21.3±0.3°, and 19.3±0.3° two theta. In some cases, the XRPD pattern further comprises at least one peak, at least two peaks, at least three peaks, or at least four peaks selected from 17.5±0.3°, 15.4±0.3°, 21.6±0.3°, or 5.8±0.3° two theta. In some cases, the XRPD pattern further comprises at least one peak, at least two peaks, or at least three peaks selected from 16.3±0.3°, 21.9±0.3°, or 23.9±0.3° two theta. In some cases, the XRPD pattern further comprises at least one peak, comprises at least two peaks, at least three peaks, at least four peaks, at least five peaks, or at least six peaks selected from 17.5±0.3°, 15.4±0.3°, 21.6±0.3°, 5.8±0.3°, 16.3±0.3°, 21.9±0.3°, or 23.9±0.3° two theta.

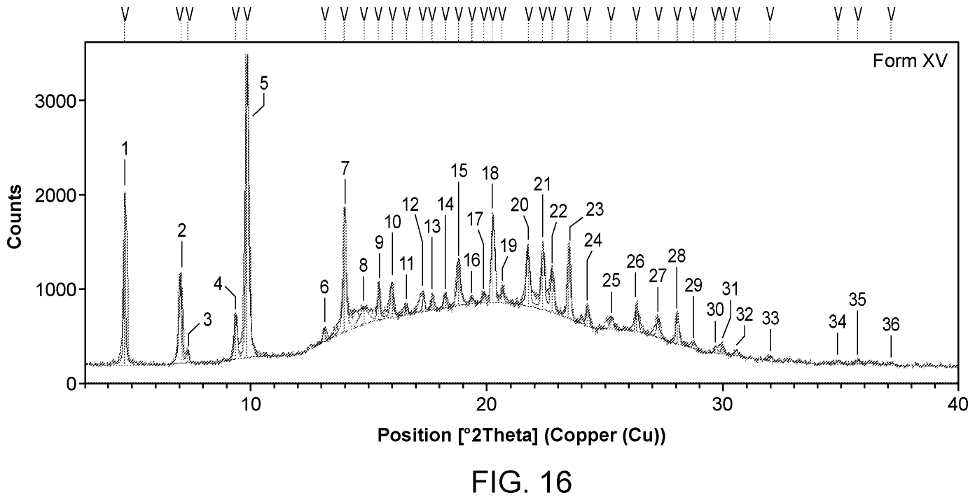

In some embodiments, polymorphic Form XV may be characterized by an x-ray powder diffraction pattern shown in FIG. 16. The crystalline form of Form XV of the compound of Formula (III) can be characterized by an XRPD pattern comprising major peaks at 9.8±0.3°, 4.7±0.3°, and 14.0±0.3° two theta. In some cases, the XRPD pattern further comprises at least one peak, at least two peaks, at least three peaks, or at least four peaks selected from 20.2±0.3°, 7.1±0.3°, 23.4±0.3°, or 22.4±0.3° two theta. In some cases, the XRPD pattern further comprises at least one peak, at least two peaks, or at least three peaks selected from 21.7±0.3°, 22.7±0.3°, or 18.8±0.3° two theta. In some cases, the XRPD pattern further comprises at least one peak, comprises at least two peaks, at least three peaks, at least four peaks, at least five peaks, or at least six peaks selected from 20.2±0.3°, 7.1±0.3°, 23.4±0.3°, 22.4±0.3°, 21.7±0.3°, 22.7±0.3°, or 18.8±0.3° two theta.

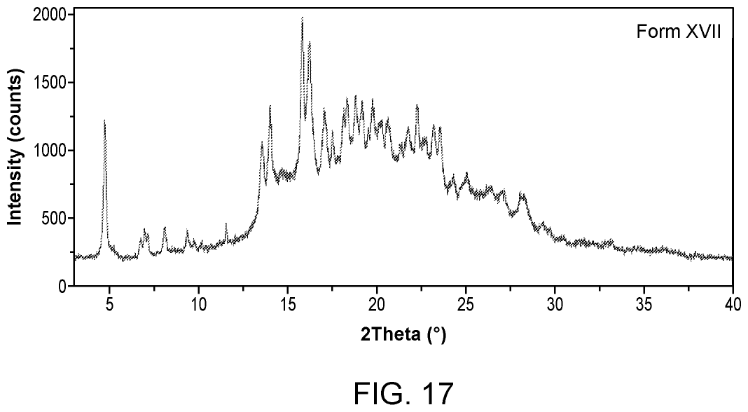

In some embodiments, polymorphic Form XVII may be characterized by an x-ray powder diffraction pattern shown in FIG. 17.

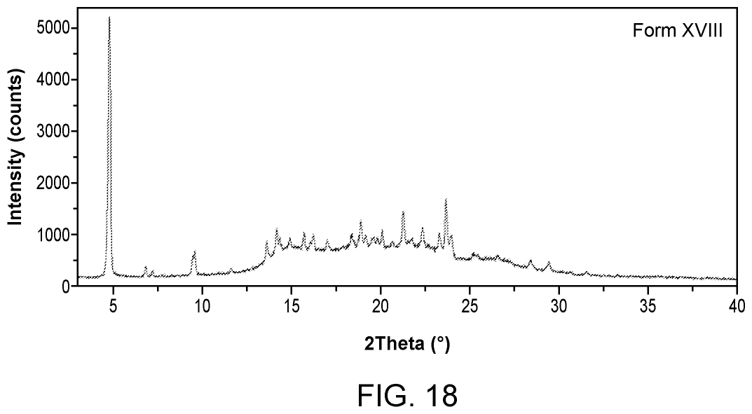

In some embodiments, polymorphic Form XVIII may be characterized by an x-ray powder diffraction pattern shown in FIG. 18.

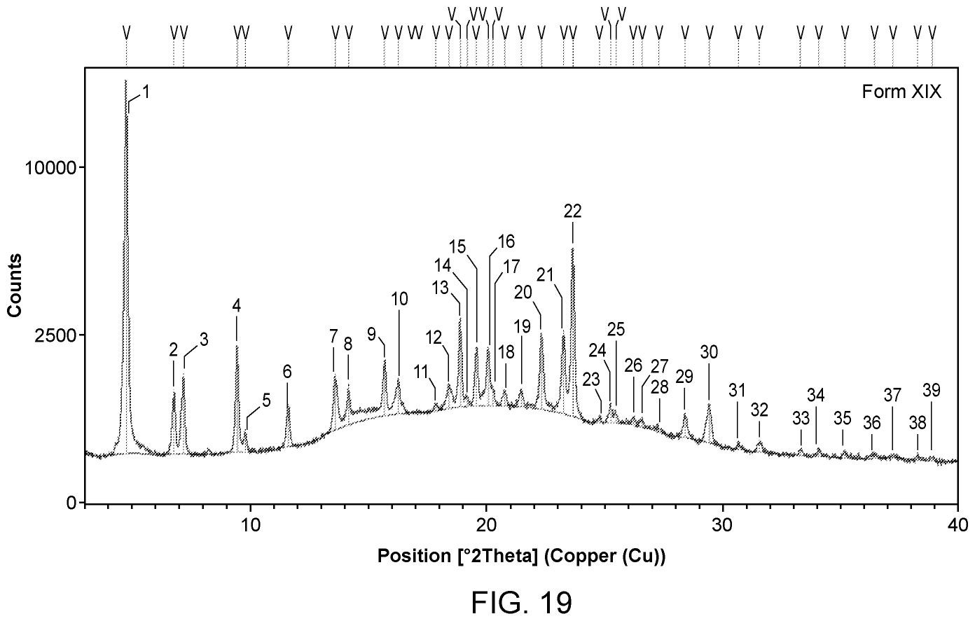

In some embodiments, polymorphic Form XIX may be characterized by an x-ray powder diffraction pattern shown in FIG. 19. The crystalline form of Form XIX of the compound of Formula (III) can be characterized by an XRPD pattern comprising major peaks at 4.7±0.3°, 23.6±0.3°, and 18.9±0.3° two theta. In some cases, the XRPD pattern further comprises at least one peak, at least two peaks, at least three peaks, or at least four peaks selected from 9.4±0.3°, 23.3±0.3°, 22.3±0.3°, or 20.1±0.3° two theta. In some cases, the XRPD pattern further comprises at least one peak, at least two peaks, or at least three peaks selected from 19.6±0.3°, 7.1±0.3°, or 15.7±0.3° two theta. In some cases, the XRPD pattern further comprises at least one peak, comprises at least two peaks, at least three peaks, at least four peaks, at least five peaks, or at least six peaks selected from 9.4±0.3°, 23.3±0.3°, 22.3±0.3°, 20.1±0.3°, 19.6±0.3°, 7.1±0.3°, or 15.7±0.3° two theta.

In some embodiments, a composition comprises endoxifen as at least 0.1%, at least 0.2%, at least 0.3%, at least 0.4%, at least 0.5%, at least 1%, at least 5%, at least 10%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, at least 99.99%, or 100% of a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, wt/wt of total endoxifen in the composition. In some embodiments, a composition for treating various cancers comprises ≥90% of a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, wt/wt of the total endoxifen in the composition. In another embodiment, the composition comprises ≥95% of a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, wt/wt of the total endoxifen in the composition. In some embodiments, the composition comprises ≥96%, ≥97%, ≥98%, ≥99%, or ≥99.5% of a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, wt/wt of the total endoxifen in the composition. When a particular percentage by weight of endoxifen is a single polymorphic form, the remainder of endoxifen in the composition may be some combination of amorphous endoxifen and/or one or more polymorphic forms of endoxifen excluding the single polymorphic form. When the polymorphic endoxifen is defined as one particular form of endoxifen, the remainder may be made up of amorphous endoxifen and/or one or more polymorphic forms other than the particular form specified. Examples of single polymorphic forms include Form I, Form II, Form III, Form IV, Form V, Form VI, Form VI, Form VIII, Form IX, Form X, Form XI, Form XII, Form XIII, Form XIV, Form XV, Form XVII, Form XVIII, and Form XIX of endoxifen, as well as descriptions of a single polymorphic form characterized by one or more properties as described herein.

In other embodiments, a composition comprising endoxifen comprises 0.01% to 20%, 0.05% to 15%, or 0.1% to 10% of a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, wt/wt or w/v of the composition. In at least one embodiment, the composition comprising endoxifen comprises 0.01% to 20% of a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, wt/wt or w/v of the composition. In various other embodiments, the composition comprising endoxifen comprises 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, or 20% of a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, wt/wt of the composition. In an aspect, a composition comprising a single polymorphic Form of endoxifen, such as Form I, Form II, Form III, Form IV, Form V, Form VI, Form VI, Form VIII, Form IX, Form X, Form XI, Form XII, Form XIII, Form XIV, Form XV, Form XVII, Form XVIII, or Form XIX, further comprises a second polymorphic Form of endoxifen.

In some embodiments, an endoxifen composition may comprise no more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, or 20% (wt/wt) of E-endoxifen, relative to total endoxifen. In some embodiments, an endoxifen composition may comprise no less than 99.9%, 99%, 98%, 97%, 96%, 95%, 90%, 85%, 80%, or 70% (wt/wt) of Z-endoxifen, relative to total endoxifen. In some embodiments, an endoxifen composition may comprise no more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, or 20% (wt/wt) of impurities, relative to Z-endoxifen. In some embodiments, an endoxifen composition may comprise no less than 99.9%, 99%, 98%, 97%, 96%, 95%, 90%, 85%, 80%, or 70% (wt/wt) of Z-endoxifen, relative to total composition.

### High Dose Formulations

High dose endoxifen formulations, such as enteric-resistant delayed release high dose endoxifen compositions, may present challenges due to the relatively high percent active pharmaceutical ingredient (% API) present in the formulation. Endoxifen formulations with high percent endoxifen relative to total composition fill weight (e.g., at least about 4% endoxifen, at least about 10% endoxifen, at least about 15% endoxifen, or from about 15% to about 20% endoxifen) may exhibit increased reactivity with an encapsulating layer (e.g., a coating or a capsule) compared to lower dose formulations (e.g., less than about 4% endoxifen). For example, a high dose endoxifen composition comprising at least about 4% endoxifen, by weight, relative to total fill weight may exhibit noticeable crosslinking with the surrounding capsule. Such reactivity (e.g., crosslinking) may negatively impact the bioavailability of endoxifen upon administration of the composition. For example, crosslinking between endoxifen and an enteric-resistant delayed release capsule encapsulating the endoxifen may inhibit dissolution of the capsule upon oral administration, preventing release of endoxifen. Reactivity of high dose endoxifen compositions may be mitigated by pharmaceutical excipients added to the endoxifen composition.

A high dose endoxifen composition of the present disclosure (e.g., an enteric-resistant delayed release high dose endoxifen formulation) may comprise an amount of (Z)-endoxifen formulated as a dosage form, such as a tablet or capsule. In some embodiments, a single high dose endoxifen dosage form (e.g., a single capsule or a single tablet) may comprise about 10 mg, about 12 mg, about 15 mg, about 16 mg, about 20 mg, about 22 mg, about 24 mg, about 25 mg, about 28 mg, about 30 mg, about 32 mg, about 35 mg, about 36 mg, about 38 mg, about 40 mg, about 42 mg, about 45 mg, about 50 mg, about 60 mg, about 70 mg, about 80 mg, about 90 mg, or about 100 mg of (Z)-endoxifen. In some embodiments, a single high dose endoxifen dosage form may comprise from about 10 mg to about 100 mg, from about 10 mg to about 80 mg, from about 10 mg to about 50 mg, from about 20 mg to about 50 mg, from about 30 mg to about 50 mg, or from about 10 mg to about 30 mg of (Z)-endoxifen.

In some embodiments, a high dose endoxifen composition may comprise from about 4% to about 40%, from about 5% to about 40%, from about 8% to about 40%, from about 10% to about 40%, from about 12% to about 40%, from about 13% to about 40%, from about 14% to about 40%, from about 15% to about 40%, from about 16% to about 40%, from about 17% to about 40%, from about 18% to about 40%, from about 4% to about 30%, from about 5% to about 30%, from about 8% to about 30%, from about 10% to about 30%, from about 12% to about 30%, from about 13% to about 30%, from about 14% to about 30%, from about 15% to about 30%, from about 16% to about 30%, from about 17% to about 30%, from about 18% to about 30%, from about 4% to about 25%, from about 5% to about 25%, from about 8% to about 25%, from about 10% to about 25%, from about 12% to about 25%, from about 13% to about 25%, from about 14% to about 25%, from about 15% to about 25%, from about 16% to about 25%, from about 17% to about 25%, from about 18% to about 25%, from about 4% to about 22%, from about 5% to about 22%, from about 8% to about 22%, from about 10% to about 22%, from about 12% to about 22%, from about 13% to about 22%, from about 14% to about 22%, from about 15% to about 22%, from about 16% to about 22%, from about 17% to about 22%, from about 18% to about 22%, from about 4% to about 20%, from about 5% to about 20%, from about 8% to about 20%, from about 10% to about 20%, from about 12% to about 20%, from about 13% to about 20%, from about 14% to about 20%, from about 15% to about 20%, from about 16% to about 20%, from about 17% to about 20%, from about 18% to about 20% (Z)-endoxifen, by weight, of the total fill weight of the composition,

A disintegrant may be included in a high dose endoxifen composition of the present disclosure to inhibit crosslinking between endoxifen and an encapsulating layer (e.g., capsule). In some embodiments, a disintegrant may comprise croscarmellose sodium, agar, alginic acid, calcium carbonate, microcrystalline cellulose, crospovidone, polacrilin potassium, sodium starch glycolate, potato or tapioca starch, pre-gelatinized starch, other starches, clays, other algins, other celluloses, gums, or a combination thereof. For example, croscarmellose sodium may be included in a high dose endoxifen composition to inhibit crosslinking between endoxifen and an enteric-resistant delayed release capsule encapsulating the endoxifen composition.

The disintegrant (e.g., croscarmellose sodium) may be included in a high dose endoxifen composition of the present disclosure at an amount of at least about 0.1%, at least about 0.5%, at least about 0.8%, at least about 1.0%, at least about 1.2%, at least about 1.4%, at least about 1.5%, at least about 1.6%, at least about 1.8%, at least about 2.0%, at least about 2.2%, at least about 2.4%, at least about 2.5%, at least about 2.6%, at least about 2.8%, or at least about 2.9%, by weight, of the total fill weight of the composition. For example, a high dose endoxifen composition may comprise at least about 2% croscarmellose sodium, by weight, of the total fill weight of the composition.

In some embodiments, the disintegrant (e.g., croscarmellose sodium) may be included in a high dose endoxifen composition of the present disclosure at an amount of from about 0.1% to about 10%, from about 1.0% to about 10%, from about 1.5% to about 10%, from about 2.0% to about 10%, from about 2.5% to about 10%, from about 2.6% to about 10%, from about 2.8% to about 10%, from about 2.9% to about 10%, from about 0.1% to about 8.0%, from about 1.0% to about 8.0%, from about 1.5% to about 8.0%, from about 2.0% to about 8.0%, from about 2.5% to about 8.0%, from about 2.6% to about 8.0%, from about 2.8% to about 8.0%, from about 2.9% to about 8.0%, from about 0.1% to about 5.0%, from about 1.0% to about 5.0%, from about 1.5% to about 5.0%, from about 2.0% to about 5.0%, from about 2.5% to about 5.0%, from about 2.6% to about 5.0%, from about 2.8% to about 5.0%, from about 2.9% to about 5.0%, from about 0.1% to about 4.0%, from about 1.0% to about 4.0%, from about 1.5% to about 4.0%, from about 2.0% to about 4.0%, from about 2.5% to about 4.0%, from about 2.6% to about 4.0%, from about 2.8% to about 4.0%, from about 2.9% to about 4.0%, from about 0.1% to about 3.0%, from about 1.0% to about 3.0%, from about 1.5% to about 3.0%, from about 2.0% to about 3.0%, from about 2.5% to about 3.0%, from about 2.6% to about 3.0%, from about 2.8% to about 3.0%, or from about 2.9% to about 3.0%, by weight, of the total fill weight of the composition. For example, a high dose endoxifen composition may comprise from about 1% to about 4% croscarmellose sodium, by weight, of the total fill weight of the composition.

In some embodiments, a filler may be included in a high dose endoxifen composition of the present disclosure. Examples of fillers include, but are not limited to, microcrystalline cellulose, talc, calcium carbonate (e.g., granules or powder), sugars such as dextrose, sucrose, lactose, a salt such as calcium carbonate, calcium phosphate, sodium carbonate, sodium phosphate, starches, powdered cellulose, cellulosic bases such as methyl cellulose, carboxymethyl cellulose dextrates, kaolin, mannitol, silicic acid, sorbitol, starch, pregelatinized starch, and mixtures thereof.

The filler (e.g., microcrystalline cellulose) may be included in a high dose endoxifen composition of the present disclosure at an amount of from about 10% to about 99%, from about 20% to about 99%, from about 30% to about 99%, from about 40% to about 99%, from about 50% to about 99%, from about 60% to about 99%, from about 70% to about 99%, from about 75% to about 99%, from about 10% to about 97%, from about 20% to about 97%, from about 30% to about 97%, from about 40% to about 97%, from about 50% to about 97%, from about 60% to about 97%, from about 70% to about 97%, from about 75% to about 97%, from about 10% to about 95%, from about 20% to about 95%, from about 30% to about 95%, from about 40% to about 95%, from about 50% to about 95%, from about 60% to about 95%, from about 70% to about 95%, from about 75% to about 95%, from about 10% to about 90%, from about 20% to about 90%, from about 30% to about 90%, from about 40% to about 90%, from about 50% to about 90%, from about 60% to about 90%, from about 70% to about 90%, from about 75% to about 90%, from about 10% to about 80%, from about 20% to about 80%, from about 30% to about 80%, from about 40% to about 80%, from about 50% to about 80%, from about 60% to about 80%, from about 70% to about 80%, or from about 75% to about 80%, by weight, of the total fill weight of the composition. For example, a high dose endoxifen composition may comprise from about 50% to about 95% microcrystalline cellulose, by weight, of the total fill weight of the composition.

In some embodiments, a lubricant may be included in a high dose endoxifen composition of the present disclosure. Examples of lubricants include, but are not limited to, magnesium stearate, calcium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and combinations thereof. Additional lubricants include, for example, a syloid silica gel (e.g., AEROSIL® 200, manufactured by W.R. GRACE® Co. of Baltimore, Md.), a coagulated aerosol of synthetic silica (e.g., marketed by DEGUSSA® Co. of Plano, Tex.), a fumed silica (e.g., CAB-O-SIL®, a pyrogenic silicon dioxide product sold by CABOT® Co. of Boston, Mass.), a silicon fluid (e.g., Q7-9120 manufactured by DOW CORNING®), and combinations thereof.

The lubricant (e.g., magnesium stearate) may be included in a high dose endoxifen composition of the present disclosure at an amount of at least about 0.1%, at least about 0.2%, at least about 0.3%, at least about 0.4%, at least about 0.5%, at least about 0.5%, at least about 0.6%, at least about 0.8%, at least about 0.8%, or at least about 1.0%, by weight, of the total fill weight of the composition. For example, a high dose endoxifen composition may comprise at least about 0.8% magnesium stearate, by weight, of the total fill weight of the composition.

In some embodiments, the lubricant (e.g., magnesium stearate) may be included in a high dose endoxifen composition of the present disclosure at an amount of from about 0.1% to about 5.0%, from about 0.2% to about 5.0%, from about 0.3% to about 5.0%, from about 0.4% to about 5.0%, from about 0.5% to about 5.0%, from about 0.6% to about 5.0%, from about 0.7% to about 5.0%, from about 0.8% to about 5.0%, from about 0.9% to about 5.0%, from about 1.0% to about 5.0%, from about 0.1% to about 4.0%, from about 0.2% to about 4.0%, from about 0.3% to about 4.0%, from about 0.4% to about 4.0%, from about 0.5% to about 4.0%, from about 0.6% to about 4.0%, from about 0.7% to about 4.0%, from about 0.8% to about 4.0%, from about 0.9% to about 4.0%, from about 1.0% to about 4.0%, from about 0.1% to about 3.0%, from about 0.2% to about 3.0%, from about 0.3% to about 3.0%, from about 0.4% to about 3.0%, from about 0.5% to about 3.0%, from about 0.6% to about 3.0%, from about 0.7% to about 3.0%, from about 0.8% to about 3.0%, from about 0.9% to about 3.0%, from about 1.0% to about 3.0%, from about 0.1% to about 2.0%, from about 0.2% to about 2.0%, from about 0.3% to about 2.0%, from about 0.4% to about 2.0%, from about 0.5% to about 2.0%, from about 0.6% to about 2.0%, from about 0.7% to about 2.0%, from about 0.8% to about 2.0%, from about 0.9% to about 2.0%, from about 1.0% to about 2.0%, from about 0.1% to about 1.5%, from about 0.2% to about 1.5%, from about 0.3% to about 1.5%, from about 0.4% to about 1.5%, from about 0.5% to about 1.5%, from about 0.6% to about 1.5%, from about 0.7% to about 1.5%, from about 0.8% to about 1.5%, from about 0.9% to about 1.5%, from about 1.0% to about 1.5%, from about 0.1% to about 1.2%, from about 0.2% to about 1.2%, from about 0.3% to about 1.2%, from about 0.4% to about 1.2%, from about 0.5% to about 1.2%, from about 0.6% to about 1.2%, from about 0.7% to about 1.2%, from about 0.8% to about 1.2%, from about 0.9% to about 1.2%, or from about 1.0% to about 1.2%, by weight, of the total fill weight of the composition. For example, a high dose endoxifen composition may comprise from about 0.5% to about 2% magnesium stearate, by weight, of the total fill weight of the composition.

Additional agents that may be included in the high dose endoxifen compositions of the present disclosure include binders, fillers, lubricants, and other pharmaceutically acceptable excipients. Binders suitable for use in the pharmaceutical compositions provided herein include, but are not limited to, sucrose, starches such as corn starch, potato starch, or starches such as starch paste, pregelatinized starch, and starch 1500, PEG 6000, methocel, WALOCEL® HM, LUVITEC®, caparolactam, AVICEL®, SMCC, UNI-PURE®, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL® PH 101, AVICEL® PH 103 AVICEL® RC 581, AVICEL® PH 105 (available from FMC Corporation, American Viscose Division, Avicel Sales, Marcus Hook, Pa.), and mixtures thereof. In some embodiments, the binder is a mixture of microcrystalline cellulose and sodium carboxymethyl cellulose. Suitable anhydrous or low moisture excipients or additives include AVICEL® PH 103 and Starch 1500 LM.

An example of a high dose endoxifen composition may comprise from about 10% to about 30% (Z)-endoxifen by weight, from about 1% to about 5% croscarmellose sodium by weight, from about 60% to about 95% microcrystalline cellulose by weight, and from about 0.5% to about 3% magnesium stearate by weight, relative to total fill weight of the composition. The composition may be encapsulated in an enteric-resistant delayed release capsule, such as a capsule comprising from about 85% to about 97% hydroxypropyl methylcellulose and from about 3% to about 10% gellan gum. In some embodiments, the high dose endoxifen formulation may be formulated in a dosage form comprising about 10 mg, about 20 mg, about 40 mg, or about 80 mg of (Z)-endoxifen per dosage unit (e.g., per capsule).

A further example of a high dose endoxifen composition may comprise from about 15% to about 20% (Z)-endoxifen by weight, from about 2% to about 4% croscarmellose sodium by weight, from about 70% to about 80% microcrystalline cellulose by weight, and from about 0.5% to about 2% magnesium stearate by weight, relative to total fill weight of the composition. The composition may be encapsulated in an enteric-resistant delayed release capsule, such as a capsule comprising from about 85% to about 97% hydroxypropyl methylcellulose and from about 3% to about 7% gellan gum. In some embodiments, the high dose endoxifen formulation may be formulated in a dosage form comprising about 10 mg, about 20 mg, about 40 mg, or about 80 mg of (Z)-endoxifen per dosage unit (e.g., per capsule).

### Enteric-Resistant Delayed Release Endoxifen Compositions

The endoxifen compositions described herein may be formulated as enteric-resistant delayed release formulations for oral delivery. An enteric-resistant delayed release endoxifen formulation may be resistant to dissolution in the acidic environment of the stomach following oral administration, slowing or preventing release of endoxifen in the stomach, and may readily dissolve in the less acidic environment of the intestines, releasing most of the endoxifen in the intestines. The enteric-resistant delayed release formulations of the present disclosure may facilitate delivery of (Z)-endoxifen by protecting the (Z)-endoxifen from the acidic environment of the stomach and preventing isomerization into (E)-endoxifen.

Enteric-resistant and delayed release properties of an endoxifen composition may be assessed using a dissolution assay, which may be performed using a variety of techniques. In some embodiments, dissolution properties of an endoxifen composition may be quantified using the dissolution assay described in TABLE 4 of EXAMPLE 2. An endoxifen composition, such as an endoxifen formulation encapsulated in an enteric-resistant delayed release capsule, may be placed in an acidic solution comprising a pH of less than about 2 for about 120 minutes, stirring at a temperature of about 37±0.5° C. Dissolution rates under acidic conditions, mimicking the stomach environment, may be assessed by measuring the concentration of endoxifen in the solution at multiple timepoints (e.g., at 30 minutes, 60 minutes, 90 minutes, 120 minutes, or combinations thereof). After about 120 minutes, the acidic solution may be replaced with a buffered solution comprising a pH of about 6.8 and about 0.75% Polysorbate 80 and stirred for about 90 minutes at a temperature of about 37±0.5° C. Dissolution rates under less acidic conditions, mimicking the intestinal environment, may be assessed by measuring the concentration of endoxifen in the solution at multiple timepoints (e.g., at 15 minutes, 30 minutes, 45 minutes, 60 minutes, 90 minutes, or combinations thereof). Alternatively, dissolution rates may be measured using a US pharmacopoeia (USP) dissolution method or a Japanese pharmacopoeia (JP) dissolution method.

An enteric-resistant delayed release formulation may release no more than about 1%, no more than about 2%, no more than about 3%, no more than about 4%, no more than about 5%, no more than about 6%, no more than about 7%, no more than about 8%, no more than about 9%, no more than about 10%, no more than about 15%, no more than about 20%, no more than about 25%, no more than about 30%, no more than about 35%, or no more than about 40% of (Z)-endoxifen in the composition within about 2 hours in an acidic solution comprising a pH of less than about 2. In some embodiments, an enteric-resistant delayed release formulation may release from about 0% to about 1%, from about 0% to about 2%, from about 0% to about 3%, from about 0% to about 4%, from about 0% to about 5%, from about 0% to about 10%, from about 0% to about 15%, from about 0% to about 20%, from about 1% to about 2%, from about 1% to about 3%, from about 1% to about 4%, from about 1% to about 5%, from about 1% to about 10%, from about 1% to about 15%, from about 1% to about 20%, from about 3% to about 3%, from about 3% to about 4%, from about 3% to about 5%, from about 3% to about 10%, from about 3% to about 15%, or from about 3% to about 20% of (Z)-endoxifen in the composition within about 2 hours in an acidic solution comprising a pH of less than about 2.

In some embodiments, an enteric-resistant delayed release formulation may release no more than about 1%, no more than about 2%, no more than about 3%, no more than about 4%, no more than about 5%, no more than about 6%, no more than about 7%, no more than about 8%, no more than about 9%, no more than about 10%, no more than about 15%, no more than about 20%, no more than about 25%, no more than about 30%, no more than about 35%, or no more than about 40% of (Z)-endoxifen in the composition within about 2 hours under acidic conditions, measured as described in TABLE 4 of EXAMPLE 2. In some embodiments, an enteric-resistant delayed release formulation may release from about 0% to about 1%, from about 0% to about 2%, from about 0% to about 3%, from about 0% to about 4%, from about 0% to about 5%, from about 0% to about 10%, from about 0% to about 15%, from about 0% to about 20%, from about 1% to about 2%, from about 1% to about 3%, from about 1% to about 4%, from about 1% to about 5%, from about 1% to about 10%, from about 1% to about 15%, from about 1% to about 20%, from about 3% to about 3%, from about 3% to about 4%, from about 3% to about 5%, from about 3% to about 10%, from about 3% to about 15%, or from about 3% to about 20% of (Z)-endoxifen in the composition within about 2 hours under acidic conditions, measured as described in TABLE 4 of EXAMPLE 2.

An enteric-resistant delayed release formulation may release at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of (Z)-endoxifen in the composition within about 1.5 hours in a solution comprising a pH of about 6.8 at about 0.75% polysorbate 80. In some embodiments, an enteric-resistant delayed release formulation may release from about 50% to about 95%, from about 60% to about 95%, from about 70% to about 95%, from about 80% to about 95%, from about 85% to about 95%, from about 86% to about 95%, from about 87% to about 95%, from about 88% to about 95%, from about 89% to about 95%, from about 90% to about 95%, from about 91% to about 95%, from about 92% to about 95%, from about 93% to about 95%, from about 94% to about 95%, from about 50% to about 97%, from about 60% to about 97%, from about 70% to about 97%, from about 80% to about 97%, from about 85% to about 97%, from about 86% to about 97%, from about 87% to about 97%, from about 88% to about 97%, from about 89% to about 97%, from about 90% to about 97%, from about 91% to about 97%, from about 92% to about 97%, from about 93% to about 97%, from about 94% to about 97%, from about 95% to about 97%, from about 50% to about 99%, from about 60% to about 99%, from about 70% to about 99%, from about 80% to about 99%, from about 85% to about 99%, from about 86% to about 99%, from about 87% to about 99%, from about 88% to about 99%, from about 89% to about 99%, from about 90% to about 99%, from about 91% to about 99%, from about 92% to about 99%, from about 93% to about 99%, from about 94% to about 99%, from about 95% to about 99%, from about 50% to about 100%, from about 60% to about 100%, from about 70% to about 100%, from about 80% to about 100%, from about 85% to about 100%, from about 86% to about 100%, from about 87% to about 100%, from about 88% to about 100%, from about 89% to about 100%, from about 90% to about 100%, from about 91% to about 100%, from about 92% to about 100%, from about 93% to about 100%, from about 94% to about 100%, or from about 95% to about 100% of (Z)-endoxifen in the composition within about 1.5 hours in a solution comprising a pH of about 6.8 at about 0.75% polysorbate 80.

In some embodiments, an enteric-resistant delayed release formulation may release at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of (Z)-endoxifen in the composition within about 1.5 hours in a solution comprising a pH of about 6.8 at about 0.75% polysorbate 80, measured as described in TABLE 4 of EXAMPLE 2. In some embodiments, an enteric-resistant delayed release formulation may release from about 50% to about 95%, from about 60% to about 95%, from about 70% to about 95%, from about 80% to about 95%, from about 85% to about 95%, from about 86% to about 95%, from about 87% to about 95%, from about 88% to about 95%, from about 89% to about 95%, from about 90% to about 95%, from about 91% to about 95%, from about 92% to about 95%, from about 93% to about 95%, from about 94% to about 95%, from about 50% to about 97%, from about 60% to about 97%, from about 70% to about 97%, from about 80% to about 97%, from about 85% to about 97%, from about 86% to about 97%, from about 87% to about 97%, from about 88% to about 97%, from about 89% to about 97%, from about 90% to about 97%, from about 91% to about 97%, from about 92% to about 97%, from about 93% to about 97%, from about 94% to about 97%, from about 95% to about 97%, from about 50% to about 99%, from about 60% to about 99%, from about 70% to about 99%, from about 80% to about 99%, from about 85% to about 99%, from about 86% to about 99%, from about 87% to about 99%, from about 88% to about 99%, from about 89% to about 99%, from about 90% to about 99%, from about 91% to about 99%, from about 92% to about 99%, from about 93% to about 99%, from about 94% to about 99%, from about 95% to about 99%, from about 50% to about 100%, from about 60% to about 100%, from about 70% to about 100%, from about 80% to about 100%, from about 85% to about 100%, from about 86% to about 100%, from about 87% to about 100%, from about 88% to about 100%, from about 89% to about 100%, from about 90% to about 100%, from about 91% to about 100%, from about 92% to about 100%, from about 93% to about 100%, from about 94% to about 100%, or from about 95% to about 100% of (Z)-endoxifen in the composition within about 1.5 hours in a solution comprising a pH of about 6.8 at about 0.75% polysorbate 80, measured as described in TABLE 4 of EXAMPLE 2.

In some embodiments, an endoxifen formulation (e.g., a high dose oral endoxifen formulation) is in the form of solid dosage forms such as capsules, tablets, mini-tablets, beads, microbeads, granules, spheres particles, multi-particulates, and the like. The endoxifen compositions of the present disclosure can be formulated to target release in the intestines and colon. Accordingly, in some embodiments, the endoxifen compositions are in the form of enteric-resistant delayed release capsules, enteric coated delayed release tablets, enteric coated delayed release tablet-in-tablets, enteric coated delayed release tablet-in-capsules, beads-in-capsules, spheres-in capsules, and the like. For example, an endoxifen formulation may be encapsulated in an enteric-resistant delayed release capsule, such as a hydroxypropyl methylcellulose (also referred to as HPMC or Hypromellose) capsule to form an enteric-resistant delayed release endoxifen composition. In some embodiments, endoxifen (e.g., (Z)-endoxifen) may be dispersed in the endoxifen compositions homogeneously.

An enteric-resistant delayed release capsule, also referred to as an enteric-resistant delayed release capsule, may encapsulate an endoxifen formulation of the present disclosure (e.g., a high dose endoxifen formulation). In some embodiments, an enteric-resistant delayed release capsule may comprise hydroxypropyl methylcellulose, gellan gum, gelatin, hydroxypropyl methylcellulose phthalate, a coloring agent, an opacifier, or any combination thereof. An enteric-resistant delayed release capsule may comprise at least about 50%, at least about 55%, at least about 60%, at least about 65%, at least about 70%, at least about 75%, at least about 80%, at least about 85%, at least about 90%, or at least about 95% hydroxypropyl methylcellulose by weight, relative to total capsule weight (e.g., not including the endoxifen formulation filling). In some embodiments, an enteric-resistant delayed release capsule may comprise from about 50% to about 99%, from about 55% to about 99%, from about 60% to about 99%, from about 65% to about 99%, from about 70% to about 99%, from about 75% to about 99%, from about 80% to about 99%, from about 85% to about 99%, from about 50% to about 95%, from about 55% to about 95%, from about 60% to about 95%, from about 65% to about 95%, from about 70% to about 95%, from about 75% to about 95%, from about 80% to about 95%, from about 85% to about 95%, from about 50% to about 90%, from about 55% to about 90%, from about 60% to about 90%, from about 65% to about 90%, from about 70% to about 90%, from about 75% to about 90%, from about 80% to about 90%, or from about 85% to about 90% hydroxypropyl methylcellulose by weight, relative to total capsule weight.

An enteric-resistant delayed release capsule may comprise at least about 1%, at least about 2%, at least about 3%, at least about 4%, at least about 5%, at least about 6%, at least about 7%, at least about 8%, at least about 9%, or at least about 10% gellan gum, gelatin, or a combination thereof by weight, relative to total capsule weight. In some embodiments, an enteric-resistant delayed release capsule may comprise from about 1% to about 10%, from about 2% to about 10%, from about 3% to about 10%, from about 4% to about 10%, from about 5% to about 10%, from about 1% to about 9%, from about 2% to about 9%, from about 3% to about 9%, from about 4% to about 9%, from about 5% to about 9%, from about 1% to about 8%, from about 2% to about 8%, from about 3% to about 8%, from about 4% to about 8%, from about 5% to about 8%, from about 1% to about 7%, from about 2% to about 7%, from about 3% to about 7%, from about 4% to about 7%, from about 5% to about 7%, from about 1% to about 6%, from about 2% to about 6%, from about 3% to about 6%, from about 4% to about 6%, from about 5% to about 6%, from about 1% to about 5%, from about 2% to about 5%, from about 3% to about 5%, or from about 4% to about 5% gellan gum, gelatin, or a combination thereof by weight, relative to total capsule weight.

Plasticizers may be added to control the softness or pliability of oral dosage forms such as shell of a capsule, caplet, or a tablet and thus, may improve the mechanical properties of the pH-sensitive materials of the capsules or coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol. In another embodiment, the plasticizer is 1% to 10%, such as 3% to 5% (wt/wt), of the composition.

Examples of glidants include, but are not limited to, colloidal silicone dioxide, cellulose, calcium phosphate, di or tri-basic and the like.

Compositions formulated for oral delivery as disclosed herein, for example, tablets, caplets, and capsules, may be coated with one or more enteric coating agent, control release agent or film forming agent to control or delay disintegration and absorption of the compositions comprising endoxifen or salts thereof in the gastrointestinal tract and thereby provide a sustained action over a longer period of time. Accordingly, in some embodiments, the tablet can be an enteric tablet, the caplet can be an enteric caplet, or the capsule can be an enteric capsule. The enteric tablets, enteric caplets, or enteric capsules of the present disclosure may be prepared by techniques known in the art.

Pharmaceutical preparations disclosed herein may comprise a control release agent. Examples of control release agent suitable for use include, without limitation, pH-dependent polymers, acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax; polyethoxylated castor oil derivatives, hydrogenated oils, glyceryl mono-, di-tribenates, glyceryl monostearate, glyceryl distearate, long chain alcohols, such as stearyl alcohol, cetyl alcohol, and polyethylene glycol; and mixtures thereof. In some embodiments, a time delay material such as glyceryl monostearate or glyceryl distearate may be used. In other embodiments, the controlled release reagent is a digestible waxy substance such as hard paraffin wax.

In some embodiments, a pharmaceutical composition comprising endoxifen (e.g., Z-endoxifen) may be formulated as a sustained release composition. In some embodiments, a pharmaceutical composition comprising a phosphoinositide 3-kinase inhibitor (e.g., alpelisib) may be formulated as a sustained release composition. Sustained release agent present in a sustained release composition of the present disclosure may be any sustained release agent known in the art to slow the release of a hydrophobic drug such as (Z)-endoxifen or a polymorph or a salt thereof.

Examples of sustained release agents include cellulosic ethers, gums, acrylic resins such as polymers and copolymers of acrylic acid, methacrylic acid, methyl acrylate, methyl methylacrylate, and combinations thereof, polyvinyl pyrrolidine, and protein-derived compounds. Examples of cellulosic ethers include hydroxyalkyl celluloses, hydroxyethyl celluloses, hydroxypropyl celluloses, hydroxypropyl methylcelluloses (HPMC or Hypromellose, for example Nos. 2208, 2906, 2910), hydroxypropyl methylcellulose phthalate (HPMCP or Hypromellose phthalate), carboxyalkyl celluloses, and carboxymethyl celluloses. In some embodiments, the at least one sustained release agent is a pH sustained release agent such as acid insoluble polymers which become increasingly soluble and permeable above pH 5.0 but remaining impermeable below pH 5.0. Such controlled release polymers target upper small intestines and/or colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers, including those available commercially from EVONIK® or ROHM® (e.g., EUDRAGIT® sustained release polymers EUDRAGIT® RL (high permeability), EUDRAGIT® RS (low permeability) and EUDRAGIT® NM 30D (low permeability))—alone or in any combination thereof to achieve the desired permeability for sustained release.

In some embodiments, compositions may comprise one or more of pH-dependent polymers such as acid insoluble polymers. The pH-dependent polymers become increasingly permeable above pH 5.0 but are impermeable at pH below 5.0 whereas acid insoluble polymers become soluble in neutral to weakly alkaline conditions. Such control release polymers target upper small intestines and colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (commercially available under the tradename EUDRAGIT® L and EUDRAGIT® S from Rohm America Inc., Piscataway, NJ as a powder or a 30% aqueous dispersion; or under the tradename EASTACRYL®, from Eastman Chemical Co., Kingsport, TN, as a 30% dispersion). Additional examples include EUDRAGIT® L100-55, EUDRAGIT® L30D-55, EUDRAGIT® L100, EUDRAGIT® L100 12,5, EUDRAGIT® S100, EUDRAGIT® S12,5, EUDRAGIT® FS 30D, EUDRAGIT® E100, EUDRAGIT® E 12,5, and EUDRAGIT® PO. In at least one embodiment, the composition comprises EUDRAGIT® L100-55. EUDRAGIT® RS and RL and EUDRAGIT® NE and NM are also useful polymers for the purpose of this disclosure. In some embodiments, the composition comprises EUDRAGIT® L30D 55. In another embodiment, the preparation comprises EUDRAGIT® FS 30D. One of skill in the art will recognize that at least some acid insoluble polymers listed herein will also be biodegradable.

Commercially available delayed release capsules such as those available from Capsugel (e.g., VCAPS® Plus enteric capsules), can be used to prepared enteric-resistant delayed release capsules and are encompassed in the present disclosure. In some embodiments, the enteric delayed release capsules can be non-animal-based capsules, such as a Hypromellose capsule (for example, commercially available self-gelling VCAPS®, VCAPS® Plus, VCAPS® Enteric, other enteric capsules made using XCELLODOSE®, DRCAPS®, Encap Colonic Delivery (ENCODE), and ENTRINSIC™ drug delivery technology from CAPSUGEL®). Other technologies known in the art and available commercially (for example, QUALICAPS®, USA, NutraScience, USA, etc.) for the formulating enteric forms of oral solid dosage forms can also be utilized. For example, the endoxifen formulations of the present disclosure may be encapsulated in a DRCAPS® enteric-resistant delayed release capsule. In at least one embodiment, the capsule is an API-in-capsule, meaning that the (Z)-endoxifen free base or salts thereof is filled neat into the capsule. In such API-in-capsule oral dosage forms, the active ingredient, (Z)-endoxifen or salts thereof can be free flowing powders or micronized powders. When the dosage form is a capsule, in at least one embodiment, the capsule can be a seamless capsule or a banded capsule.

An oral dosage form can be of any shape suitable for oral administration, such as spherical (0.05-5 mL), oval (0.05-7 mL), ellipsoidal, pear (0.3-5 mL), cylindrical, cubic, regular and/or irregular shaped. An oral dosage form may be of any size suitable for oral administration, for example, size 0, size 2, and the like.

An example of an enteric-resistant delayed release endoxifen composition may comprise an enteric-resistant delayed release capsule comprising from about from about 65% to about 95% hydroxypropyl methylcellulose by weight and from about 3% to about 10% gellan gum by weight, relative to total unfilled capsule weight, encapsulating an endoxifen formulation comprising from about 10% to about 30% (Z)-endoxifen by weight, from about 1% to about 5% croscarmellose sodium by weight, from about 60% to about 95% microcrystalline cellulose by weight, and from about 0.5% to about 3% magnesium stearate by weight, relative to total fill weight of the composition. In some embodiments, the enteric-resistant delayed release endoxifen composition may be formulated in a dosage form comprising about 10 mg, about 20 mg, about 40 mg, or about 80 mg of (Z)-endoxifen per capsule.

### Methods of Treatment

An endoxifen composition of the present disclosure (e.g., a high dose enteric-resistant delayed release endoxifen formulation) may be used in a method of treating a disease or disorder in a subject, such as a human subject. In some embodiments, the subject may be a female subject. For example, the subject may be a pre-menopausal (e.g., less than about 55 years of age) female subject. In some embodiments, the disease may be a cancer, a hormone-dependent breast disorder, or a hormone-dependent reproductive tract disorder. In some embodiments, the cancer is breast cancer, cervical cancer, ovarian cancer, endometrial cancer, uterine cancer, vaginal cancer, vulvar cancer, melanoma, colorectal cancer, gastric cancer, neuroblastoma, pancreatic cancer, esophageal cancer, rectal cancer, or cholangiocarcinoma. For example, the cancer may be a breast cancer such as triple negative breast cancer, ductal carcinoma in situ, lobular carcinoma in situ, invasive ductal carcinoma, or invasive lobular carcinoma. In some embodiments, the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, precocious puberty, or McCune-Albright Syndrome. In some embodiments, the compositions described herein may be used to treat a tamoxifen-resistant or tamoxifen-refractory disorder, such as a tamoxifen-resistant or tamoxifen-refractory cancer.

An enteric-resistant delayed release endoxifen composition may be administered to a subject orally such that the (Z)-endoxifen in the composition is delivered to an intestine of the subject. In some embodiments, no more than about 1%, no more than about 2%, no more than about 3%, no more than about 4%, no more than about 5%, no more than about 6%, no more than about 7%, no more than about 8%, no more than about 9%, no more than about 10%, no more than about 15%, no more than about 20%, no more than about 25%, no more than about 30%, no more than about 35%, or no more than about 40% of the (Z)-endoxifen in the composition is released in a stomach of the subject. In some embodiments, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of the (Z)-endoxifen in the composition is released in an intestine of the subject.

An enteric-resistant delayed release formulation may release no more than about 1%, no more than about 2%, no more than about 3%, no more than about 4%, no more than about 5%, no more than about 6%, no more than about 7%, no more than about 8%, no more than about 9%, no more than about 10%, no more than about 15%, no more than about 20%, no more than about 25%, no more than about 30%, no more than about 35%, or no more than about 40% of (Z)-endoxifen in the composition in the stomach of a subject following oral administration. In some embodiments, an enteric-resistant delayed release formulation may release from about 0% to about 1%, from about 0% to about 2%, from about 0% to about 3%, from about 0% to about 4%, from about 0% to about 5%, from about 0% to about 10%, from about 0% to about 15%, from about 0% to about 20%, from about 1% to about 2%, from about 1% to about 3%, from about 1% to about 4%, from about 1% to about 5%, from about 1% to about 10%, from about 1% to about 15%, from about 1% to about 20%, from about 3% to about 3%, from about 3% to about 4%, from about 3% to about 5%, from about 3% to about 10%, from about 3% to about 15%, or from about 3% to about 20% of (Z)-endoxifen in the composition in the stomach of a subject following oral administration.

An enteric-resistant delayed release formulation may release at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of (Z)-endoxifen in the composition in the intestines of a subject following oral administration. In some embodiments, an enteric-resistant delayed release formulation may release from about 50% to about 95%, from about 60% to about 95%, from about 70% to about 95%, from about 80% to about 95%, from about 85% to about 95%, from about 86% to about 95%, from about 87% to about 95%, from about 88% to about 95%, from about 89% to about 95%, from about 90% to about 95%, from about 91% to about 95%, from about 92% to about 95%, from about 93% to about 95%, from about 94% to about 95%, from about 50% to about 97%, from about 60% to about 97%, from about 70% to about 97%, from about 80% to about 97%, from about 85% to about 97%, from about 86% to about 97%, from about 87% to about 97%, from about 88% to about 97%, from about 89% to about 97%, from about 90% to about 97%, from about 91% to about 97%, from about 92% to about 97%, from about 93% to about 97%, from about 94% to about 97%, from about 95% to about 97%, from about 50% to about 99%, from about 60% to about 99%, from about 70% to about 99%, from about 80% to about 99%, from about 85% to about 99%, from about 86% to about 99%, from about 87% to about 99%, from about 88% to about 99%, from about 89% to about 99%, from about 90% to about 99%, from about 91% to about 99%, from about 92% to about 99%, from about 93% to about 99%, from about 94% to about 99%, from about 95% to about 99%, from about 50% to about 100%, from about 60% to about 100%, from about 70% to about 100%, from about 80% to about 100%, from about 85% to about 100%, from about 86% to about 100%, from about 87% to about 100%, from about 88% to about 100%, from about 89% to about 100%, from about 90% to about 100%, from about 91% to about 100%, from about 92% to about 100%, from about 93% to about 100%, from about 94% to about 100%, or from about 95% to about 100% of (Z)-endoxifen in the composition in the intestines of a subject following oral administration.

A method of treatment may comprise daily administration of an endoxifen composition to a subject. In some embodiments, from about 10 mg to about 100 mg, from about 20 mg to about 100 mg, from about 30 mg to about 100 mg, from about 40 mg to about 100 mg, from about 60 mg to about 100 mg, from about 80 mg to about 100 mg, from about 10 mg to about 90 mg, from about 20 mg to about 90 mg, from about 30 mg to about 90 mg, from about 40 mg to about 90 mg, from about 60 mg to about 90 mg, from about 80 mg to about 90 mg, from about 10 mg to about 80 mg, from about 20 mg to about 80 mg, from about 30 mg to about 80 mg, from about 40 mg to about 80 mg, from about 50 mg to about 80 mg, from about 10 mg to about 60 mg, from about 20 mg to about 60 mg, from about 30 mg to about 60 mg, from about 40 mg to about 60 mg, from about 50 mg to about 60 mg of (Z)-endoxifen are administered to a subject per day. In some embodiments, the (Z)-endoxifen may be administered in the form of an enteric-resistant delayed release capsule comprising about 10 mg, about 20 mg, about 40 mg, or about 80 mg (Z)-endoxifen per capsule.

The compositions of the present disclosure may be administered to a subject for a duration of from about 7 to about one year. In some embodiments, the composition may be administered daily. In some embodiments, the endoxifen compositions of the present disclosure are administered until the disease or condition is treated. For example, an endoxifen composition may be administered to a subject daily for up to about 6 months or until the disease or condition is treated. In another example, an endoxifen composition may be administered to a subject daily for about 28 days to about 6 months or until the disease or condition is treated. Treating the disease or condition may comprise alleviating one or more symptoms of the disease or reducing or eliminating the disease. For example, an endoxifen composition may be administered daily to a subject having a tumor until a tumor in the subject begins to shrink, reduces in size by a predetermined amount, or is no longer detectable.

Following administration, a blood plasma concentration of endoxifen may be measured in the subject. In some embodiments, following oral administration of an endoxifen composition of the present disclosure, the blood plasma concentration of endoxifen may comprise at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of endoxifen in the (Z)-isoform, by weight, relative to total endoxifen present in the blood plasma of the subject.

In some embodiments, an endoxifen composition may be administered to a subject in combination with an additional therapeutic agent. Examples of additional therapeutic agents that may be used in combination with the endoxifen formulations of the present disclosure include but are not limited to bicalutamide, enzalutamide, or an anticancer drug such as trastuzumab, antineoplastics such as capecitabine (e.g., XELODA®), carboplatin (e.g., PARAPLATIN®), cisplatin (e.g., PLATINOL®), cyclophosphamide (e.g., NEOSAR®), docetaxel (e.g., DOCEFREZ®, TAXOTERE®), doxorubicin (e.g., ADRIAMYCIN®), PEGylated liposomal doxorubicin (e.g., DOXIL®), epirubicin (e.g., ELLENCE®), fluorouracil (5-FU, e.g., ADRUCIL®), gemcitabine (e.g., GEMZAR®), methotrexate (multiple brand names), paclitaxel (e.g., TAXOL®), protein-bound paclitaxel (e.g., ABRAXANE®), vinorelbine (e.g., NAVELBINE®), eribulin (e.g., HALAVEN®), ixabepilone (e.g., IXEMPRA®), and ATP-cassette binding protein transport inhibitors.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the terms “a,” “an,” and “the” include plural reference unless the context dictates otherwise.

It is specifically understood that any numerical value cited herein includes all values from the lower value to the upper value, i.e., all possible combination of numerical values between the lowest value and the highest value enumerated are to be considered to be expressly stated in this application and the endpoint of all ranges are included within the range and independently combinable. For example, if a concentration range or beneficial range is stated as 1% to 50%, it is intended that values such as 2% to 40%, 10% to 30%, or 1% to 3% etc., are expressly enumerated in this specification. It is also to be understood that if a concentration or dose is stated as a specific value such as 1 mg or 10 mg, it is intended that it is intended to include 10% variation. As another example, a stated concentration of 20% is intended to include values ±10%. Yet another example, if a ratio of 1:10 to 10:1 is stated, then it is intended that ratios such as 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1 or from 2:5 to 3:5 etc. are specifically intended. There are only some examples of what is specifically intended. Unless specified otherwise, the values of the constituents or components of the compositions are expressed in weight percent of each ingredient in the component.

As used herein, the terms “active pharmaceutical ingredient,” “active ingredient,” “API,” “drug,” “active,” “actives,” or “therapeutic agent” may be used interchangeably to refer to the pharmaceutically active compound(s) in a pharmaceutical composition. This is in contrast to other ingredients in the compositions, such as excipients, which are substantially or completely pharmaceutically inert. A suitable API in accordance with the present disclosure is one where there is or likely may be patient compliance issues for treating a certain disease, condition, or disorder. The therapeutic agent as used herein includes the active compound and its salts, prodrugs, and metabolites. As used herein the term “drug” means a compound intended for use in diagnosis, cure, mitigation, treatment, and/or prevention of disease in man or other animals.

As used herein, “adjuvant therapy” refers to a therapy that follows a primary therapy and that is administered to subjects at risk of relapsing. Adjuvant systemic therapy in case of breast cancer or reproductive tract cancer, for example with tamoxifen, usually begins soon after primary therapy to delay recurrence, prolong survival or cure a subject.

Embodiments that reference throughout this specification to “a compound,” such as compounds of Formula (I) or Formula (II), include the polymorphic, salt, free base, co-crystal, and solvate forms of the formulas and/or compounds disclosed herein.

The terms “crystalline form,” “polymorph,” and “Form” may be used interchangeably herein and are meant to include all crystalline and amorphous forms of the compound, including, for example, polymorphs, pseudopolymorphs, salts, solvates, hydrates, unsolvated polymorphs (including anhydrates), conformational polymorphs, and amorphous forms, as well as mixtures thereof, unless a particular crystalline or amorphous form is referred to. Compound of the present disclosure include crystalline and amorphous forms of those compounds, including, for example, polymorphs, pseudopolymorphs, salts, solvates, hydrates, unsolvated polymorphs (including anhydrates), conformational polymorphs, and amorphous forms of the compounds, as well as mixtures thereof.

As used herein and in the claims, the terms “comprising,” “containing,” and “including” are inclusive, open-ended and do not exclude additional unrecited elements, compositional components, or method steps. Accordingly, the terms “comprising” and “including” encompass the more restrictive terms “consisting of” and “consisting essentially of.”

As used herein, the term “combination therapy” refers to the use of a composition described herein in combination with one or more additional treatment. Treatment in combination therapy can be any treatment such as any prophylactic agent, therapeutic agent (such as chemotherapy), radiotherapy, surgery, and the like. The combination can refer to inclusion of a therapeutic or prophylactic agent in a same composition as a composition disclosed herein (for example, in the same capsule, tablet, ointment, etc.) or in separate compositions (for example, in 2 separate capsules). The separate compositions may be in a different dosage form. The use of the terms “combination therapy” and “in combination with” does not restrict the order in which a composition described herein and prophylactic and/or therapeutic agent and/or treatment are administered to a subject in need thereof. Compositions of the present disclosure can be administered prior to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year before), concomitant with, or subsequent to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year after) administration of one or more prophylactic and/or therapeutic agent and/or treatment to a subject in thereof. Combination therapy as used herein can also refer to treatment of a subject having a single disease or multiple diseases, for example, prostate cancer in men and gynecomastia.

As used herein, the term “pharmaceutically acceptable carrier” or “carrier” means a pharmaceutically acceptable material, composition, or vehicle, such as a liquid or solid filler, diluent, excipient, solvent, or encapsulating material, involved in carrying or transporting one or more of the compounds of the present disclosure from one tissue, organ, or portion of the body or across the skin.

As used herein, the term “pharmaceutically acceptable salt” refers to any salt (e.g., obtained by reaction with an acid or a base) of a compound of the present disclosure that is physiologically tolerated in a subject (e.g., a mammal, and/or in vivo, ex-vivo, in vitro cells, tissues, or organs). A “salt” of a compound of the present disclosure may be derived from inorganic or organic acids and bases. Suitable anion salts include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide.

Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

As used herein, the term “pharmaceutical composition” means a combination of the active agent (e.g., an active pharmaceutical compound or ingredient, API) with a carrier, inert or active (e.g., a phospholipid), making the compositions particularly suitable for diagnostic or therapeutic uses in vitro, in vivo, or ex vivo.

As used herein, the terms “subject,” “patient,” “participant,” and “individual,” may be used interchangeably herein and refer to a mammal such as a human. Mammals also include pet animals such as dogs, cats, laboratory animals, such as rats, mice, and farm animals such as cows and horses. Unless otherwise specified, a mammal may be of any gender or sex.

All methods described herein can be performed in a suitable order unless otherwise indicated or otherwise clearly contradicted by context. The use of any and all examples, or exemplary language (e.g., “such as” and “the like”) is intended merely to illustrate the invention and does not pose a limitation on the scope of the invention unless otherwise claimed. No language in the specification should be construed as any indicating any non-claimed element as essential to practice of the invention as used herein.

### EXAMPLES

The invention is further illustrated by the following non-limiting examples.

### Example 1

### pH-Dependent Solubility of (Z)-Endoxifen

This example describes pH-dependent solubility of (Z)-endoxifen. (Z)-endoxifen was dissolved in solution under different pH conditions, and the solubility was measured. Since (Z)-endoxifen converts to the E-form under acidic conditions, the solubility of (Z)-endoxifen and (E)-endoxifen were measured independently, and a ratio of Z-form to E-form was determined.

TABLE 1
Solubility of (Z)-Endoxifen Under Different pH Conditions

| | Solubility (μg/mL) | | | Ratio |
|---|---|---|---|---|
| pH | Z + E form | Z-form | E-form | Z:E |
| 1.2 | 1631.85 | 1047.35 | 584.50 | 64:36 |
| 4.5 | 264.60 | 249.66 | 14.94 | 94:6 |
| 5.0 | 215.56 | 202.83 | 12.72 | 94:6 |
| 5.5 | 52.68 | 43.45 | 9.23 | 82:18 |
| 6.0 | 28.69 | 27.80 | 0.89 | 97:3 |
| 6.8 | 4.26 | 4.26 | 0 | 100:0 |
| Water | 0.78 | 0.78 | 0 | 100:0 |

As shown in TABLE 1, the ratio of Z-form to E-form was increased as pH increased, demonstrating that (Z)-endoxifen converted to (E)-endoxifen more readily under acidic conditions than at more neutral pH (e.g., pH 6.8). At pH 6.8, any Z-form to E-form equilibration was below the limit of detection. Since the active form of endoxifen is (Z)-endoxifen, this data suggested that a delivery mechanism that bypasses the acidic conditions of the stomach may enable delivery of endoxifen in its active Z-form. A decrease in the total solubility of endoxifen (Z and E forms) was also observed as the pH increased.

### Example 2

### Dissolution Assay Development

This example describes development of a dissolution assay to measure dissolution of endoxifen formulations. Since solubility of endoxifen decreases as pH becomes less acidic, as shown in EXAMPLE 1, additional experiments were performed to improve solubility of endoxifen in the pH 6.8 buffer solution. Polysorbate 80 (PS80), a nonionic surfactant, was added in varying concentrations to improve solubility and mimic an intestinal environment. Surfactants, such as polysorbate 80, create micelles in the medium that mimic the bile acid aggregates in the small intestine. These micelles facilitate the diffusion and transport of product into the medium.

TABLE 2
Solubility of (Z)-Endoxifen at pH
6.8 with and without Polysorbate 80

| Sample | Solubility (μg/mL) |
|---|---|
| (Z)-endoxifen in pH 6.8 Buffer (Control) | 8.14 |
| (Z)-endoxifen in pH 6.8 Buffer | 58.31 |
| with 0.5% Polysorbate 80 | |
| (Z)-endoxifen in pH 6.8 Buffer | 62.15 |
| with 1.0% Polysorbate 80 | |

As shown in TABLE 2, the addition of polysorbate 80 improved the solubility of endoxifen at pH 6.8.

Based on this solubility data, a dissolution method was developed for testing dissolution of encapsulated endoxifen formulations. The improved dissolution assay was developed based on a combination of the US pharmacopoeia (USP) dissolution method and the Japanese pharmacopoeia (JP) dissolution method. The media used in each of these methods is summarized in TABLE 3.

TABLE 3
Comparison of USP and JP Dissolution Method Media

| | Acid Medium | Buffer Medium |
|---|---|---|
| USP/EP | 750 mL of 0.1N hydrochloric acid | Add 250 mL of 0.20M tribasic |
| | during 2 hours | sodium phosphate during 45 minutes |
| | | (or for the specified time given in the |
| | | individual monograph) |
| JP | 2 g of sodium chloride in 700 mL of | Dissolve 1.70 g of potassium |
| | demineralized water. Add 7 mL of | dihydrogen phosphate and 1.775 g of |
| | concentrated hydrochloric acid (Merk, | disodium hydrogen phosphate |
| | 36% in water) under stirring. Complete | anhydrous in sufficient demineralized |
| | with demineralized water qsp 1000 mL. | water to make 1000 mL. |

The improved dissolution method also included either 0.5% or 0.75% polysorbate 80 in the buffer medium to mimic conditions of the intestine and increase endoxifen solubility. The improved dissolution method is summarized in TABLE 4.

TABLE 4
Improved Dissolution Method Conditions

| Parameters | Acid Stage Conditions | Buffer Stage Conditions |
|---|---|---|
| Medium | 2 g of sodium chloride in 700 mL | Dissolve 1.70 g of potassium |
| | of demineralized water. Add 7 mL | dihydrogen phosphate and 1.775 g |
| | of concentrated hydrochloric acid | of disodium hydrogen phosphate |
| | (Merk, 36% in water) under | anhydrous in sufficient |
| | stirring. Complete with | demineralized water and 0.75%¹ |
| | demineralized water qsp 1000 mL. | Polysorbate 80 to make 1000 mL. |
| Volume | 1000 mL | 1000 mL |
| Apparatus | USP type II (Paddle) | USP type II (Paddle) |
| Sinkers | Yes | Yes |
| Rotational | 75 rpm | 75 rpm |
| Speed | | |
| Pull Time | 30, 60, 90, and 120 | 15, 30, 45, 60, and 90 |
| (minutes)² | | |
| Temperature | 37 ± 0.5° C. | 37 ± 0.5° C. |

¹0.5% PS80 was tested and showed incomplete dissolution therefore, 0.75% PS80 was tested and achieved full dissolution. Hence, moving forward, 0.75% PS80 will be included in the buffer medium.
²The pull times in this table correspond to the pull times used during the development of the dissolution method. The pull times may be altered in the final dissolution method used to release the drug product.

Briefly, an encapsulated endoxifen formulation was weighted down with a sinker in a vessel and incubated at about 37° C. in 1000 mL of acid medium for 2 hours while mixing with a paddle. Samples were collected at 30, 60, 90, and 120 minutes to measure the amount of capsule and endoxifen formulation that had dissolved into the medium. After 2 hours, the acid medium was replaced with 1000 mL of buffer medium and incubated for an additional 1.5 hours. Samples were collected at 15, 30, 45, 60, and 90 minutes after buffer replacement.

Samples collected at each pull time were assayed using HPLC-UV to determine percent release of endoxifen over time. Chromatography was performed using a Waters Xselect CSH Phenyl-Hexyl 3.5 μm, 4.6 mm×150 mm column or equivalent with a flow rate of 0.8 mL/min, a run time of 18 minutes, and an injection volume of 80 μL. The column was held at about 50° C., and UV of the elution was measured at 243 nm. The autosampler was held at a temperature of 5° C. to ensure stability of the samples. A gradient between mobile phase A (10 mM ammonium formate in water) and mobile phase B (ammonium formate in methanol) was performed as shown in TABLE 5.

TABLE 5
Chromatography Gradient Conditions

| | Mobile | Mobile |
|---|---|---|
| Time (min) | phase A (%) | phase B (%) |
| 0.0 | 70 | 30 |
| 2.0 | 70 | 30 |
| 2.5 | 30 | 70 |
| 12.5 | 30 | 70 |
| 12.6 | 10 | 90 |
| 15.0 | 10 | 90 |
| 15.1 | 70 | 30 |
| 18.0 | 70 | 30 |

This improved dissolution assay was used to measure dissolution of high dose (Z)-endoxifen formulations.

### Example 3

### In Vitro Dissolution of High Dose (Z)-Endoxifen Formulations

This example describes in vitro dissolution of high dose (Z)-endoxifen formulations. A (Z)-endoxifen formulation containing 40 mg of (Z)-endoxifen (active pharmaceutical ingredient (API)), magnesium stearate, and microcrystalline cellulose was encapsulated in an enteric-resistant, delayed release capsule (DRCAPS®, CAPSUGEL®, USA) comprising Hypromellose and gellan gum. The 40 mg (Z)-endoxifen formulation contained about 18% API (versus total fill weight). Initial dissolution assays showed poor capsule disintegration, preventing release of (Z)-endoxifen. The poor capsule disintegration was speculated to be due to crosslinking of the (Z)-endoxifen API with the capsule shell. This crosslinking effect was not observed with lower dose formulations, including 1 mg, 2 mg, and 4 mg formulations, or lower % API formulations, including formulations with up to 4% (Z)-endoxifen (versus total fill weight).

To mitigate the crosslinking, croscarmellose sodium was added as a disintegrant to the 40 mg (Z)-endoxifen formulation. To test the effect of croscarmellose sodium on crosslinking between the (Z)-endoxifen API and the capsule, dissolution assays were performed as described in TABLE 4 and TABLE 5 of EXAMPLE 2. The tested formulation (V1 through V6) contained 40 mg (Z)-endoxifen, 173.78 mg microcrystalline cellulose, 6.5 mg croscarmellose sodium, and 2.23 mg magnesium stearate per capsule, for a total fill weight of 222.5 mg and 18% API. The formulation was encapsulated in an enteric-resistant, delayed release capsule (DRCAPS®, CAPSUGEL®, USA) comprising Hypromellose and gellan gum, the same capsules used for dissolution assays with the formulation lacking croscarmellose sodium.

Dissolution assays performed in the presence of 0.5% polysorbate 80 showed full disintegration of the capsule, suggesting crosslinking had been reduced or eliminated, but incomplete dissolution of (Z)-endoxifen. The results of this assay are provided in TABLE 6.

TABLE 6
Dissolution with 0.5% Polysorbate 80 in Buffer Medium

| Vessel | | V1 | V2 | V3 | V4 | V5 | V6 | Average |
|---|---|---|---|---|---|---|---|---|
| % Dissolution | 2 hours¹ | 3 | 3 | 2 | 3 | 7 | 2 | 3 |
| | 3.5 hours² | 89 | 83 | 89 | 89 | 91 | 85 | 88 |
| | Infinity | 86 | 85 | 88 | 82 | 92 | 83 | 86 |

¹2 hours in acid medium
²2 hours in acid medium + 90 minutes in buffer medium

Dissolution assays performed in the presence of 0.75% polysorbate 80 also showed full disintegration of the capsule, as well as full dissolution of (Z)-endoxifen. The results of this assay are provided in TABLE 7.

TABLE 7
Dissolution with 0.75% Polysorbate 80 in Buffer Medium

| Vessel | | V1 | V2 | V3 | V4 | V5 | V6 | Average |
|---|---|---|---|---|---|---|---|---|
| % Dissolution | 2 hours¹ | 3 | 4 | 3 | 3 | 4 | 4 | 4 |
| | 3.5 hours² | 100 | 91 | 83 | 97 | 108 | 104 | 97 |
| | Infinity | 107 | 108 | 102 | 98 | 109 | 97 | 103 |

¹2 hours in acid medium
²2 hours in acid medium + 90 minutes in buffer medium

In contrast to the high dose endoxifen formulations lacking croscarmellose sodium, which failed to dissolve in dissolution assays, the high dose endoxifen formulations with croscarmellose sodium exhibited full capsule disintegration and complete dissolution of (Z)-endoxifen. These results indicated that the addition of croscarmellose sodium mitigated crosslinking between the API and the capsule, enabling full disintegration of the capsule.

### Example 4

### Manufacture of a High Dose (Z)-Endoxifen Formulation

This example describes manufacture of a high dose (Z)-endoxifen formulation. The 40 mg (Z)-endoxifen (18% API) formulation assayed in EXAMPLE 4 containing croscarmellose sodium to prevent crosslinking between the endoxifen API and the capsule was manufactured and validated. The per-capsule formulations and percent of drug formulation (excluding capsule), by weight, are provided in TABLE 8.

TABLE 8
40 mg (Z)-Endoxifen Formulation

| Material | Per Capsule | Percent (by weight) |
|---|---|---|
| (Z)-Endoxifen | 40.00 mg | 17.98% |
| Microcrystalline | 173.78 mg | 78.10% |
| cellulose | | |
| Croscarmellose | 6.50 mg | 2.92% |
| sodium | | |
| Magnesium | 2.23 mg | 1.00% |
| stearate | | |
| Total fill weight | 222.51 mg | 100.00% |

(Z)-endoxifen, microcrystalline cellulose, and croscarmellose sodium were co-shifted and blended. The magnesium stearate was shifted separately and mixed with the endoxifen blend for 2 minutes. The final blend was filled in size 0 Hypromellose enteric-resistant, delayed release capsules (DRCAPS®, CAPSUGEL®, USA) with a fill weight of 222.5 mg±1 mg.

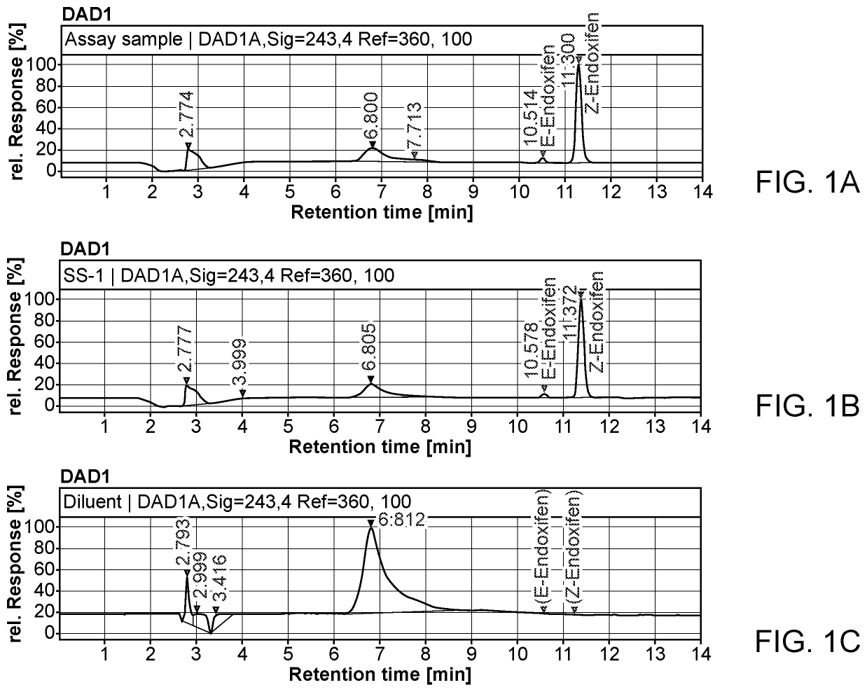

The (Z)-endoxifen formulation was assayed by HPLC to validate the composition. The contents of five (Z)-endoxifen capsules was emptied into 180 mL of diluent sonicated for 5 minutes. The dissolved sample was allowed to equilibrate to room temperature and diluted to 0.05 mg/mL (Z)-endoxifen in diluent. HPLC chromatograms of the 0.5 mg/mL (Z)-endoxifen sample (FIG. 1A) were compared to those of a 0.05 mg/mL (Z)-endoxifen reference sample (FIG. 1B) and diluent alone (FIG. 1C). The manufactured (Z)-endoxifen sample closely matched the (Z)-endoxifen reference sample.

### Example 5

### Oral Administration of a High Dose (Z)-Endoxifen Formulation

This example describes oral administration of a high dose (Z)-endoxifen formulation. A human subject is orally administered 80 mg of (Z)-endoxifen in the form of two 40 mg enteric-resistant, delayed release capsules or a single 80 mg enteric-resistant, delayed release capsule. Each 40 mg capsule contains 40 mg (Z)-endoxifen, 173.78 mg microcrystalline cellulose, 6.50 mg croscarmellose sodium, and 2.23 mg magnesium stearate encapsulated in a Hypromellose capsule, delayed release capsule. Upon oral administration, the capsule remains mostly intact in the acidic environment of the stomach. The capsule begins to dissolve as it exits the stomach and enters the intestine, releasing less than about 10% of the (Z)-endoxifen in the stomach. Once in the intestine, the capsule fully dissolves, releasing the remaining (Z)-endoxifen in the intestine. Plasma and serum levels of endoxifen in the subject show that at least 90% of endoxifen is in the active Z-form and less than 10% of endoxifen is in the E-form.

### Example 6

### Treatment of Breast Cancer Using a High Dose (Z)-Endoxifen Formulation

This example describes treatment of breast cancer using a high dose (Z)-endoxifen formulation. A patient having breast cancer is orally administered 80 mg of (Z)-endoxifen per day for up to 6 months, or until the breast cancer is treated. The 80 mg of (Z)-endoxifen is formulated as two 40 mg enteric-resistant, delayed release capsules or a single 80 mg enteric-resistant, delayed release capsule. The capsules are resistant to dissolution in the acidic environment of the stomach, such that the capsule dissolves primarily in the less acidic environment of the intestines following oral administration of the capsule to the subject. As a result, at least about 85% of the (Z)-endoxifen is released in the intestines of the subject, and no more than about 10% of the (Z)-endoxifen is released in the stomach of the subject. Following oral administration of the (Z)-endoxifen enteric-resistant, delayed release formulation, at least about 85% of endoxifen present in the blood plasma and serum of the subject is in the Z-isoform.

Oral administration of a high dose of (Z)-endoxifen eliminates, slows the spread of, shrinks, or reduces symptoms of the breast cancer, thereby treating the breast cancer.

### Example 7

### Treatment of Ovarian Cancer Using a High Dose (Z)-Endoxifen Formulation

This example describes treatment of ovarian cancer using a high dose (Z)-endoxifen formulation. A patient having ovarian cancer is orally administered 80 mg of (Z)-endoxifen per day for up to 6 months, or until the ovarian cancer is treated. The 80 mg of (Z)-endoxifen is formulated as two 40 mg enteric-resistant, delayed release capsules or a single 80 mg enteric-resistant, delayed release capsule. The capsules are resistant to dissolution in the acidic environment of the stomach, such that the capsule dissolves primarily in the less acidic environment of the intestines following oral administration of the capsule to the subject. As a result, at least about 85% of the (Z)-endoxifen is released in the intestines of the subject, and no more than about 10% of the (Z)-endoxifen is released in the stomach of the subject. Following oral administration of the (Z)-endoxifen enteric-resistant, delayed release formulation, at least about 85% of endoxifen present in the blood plasma and serum of the subject is in the Z-isoform.

Oral administration of a high dose of (Z)-endoxifen eliminates, slows the spread of, shrinks, or reduces symptoms of the ovarian cancer, thereby treating the ovarian cancer.

### Example 8

### Treatment of Cervical Cancer Using a High Dose (Z)-Endoxifen Formulation

This example describes treatment of cervical cancer using a high dose (Z)-endoxifen formulation. A patient having cervical cancer is orally administered 80 mg of (Z)-endoxifen per day for up to 6 months, or until the cervical cancer is treated. The 80 mg of (Z)-endoxifen is formulated as two 40 mg enteric-resistant, delayed release capsules or a single 80 mg enteric-resistant, delayed release capsule. The capsules are resistant to dissolution in the acidic environment of the stomach, such that the capsule dissolves primarily in the less acidic environment of the intestines following oral administration of the capsule to the subject. As a result, at least about 85% of the (Z)-endoxifen is released in the intestines of the subject, and no more than about 10% of the (Z)-endoxifen is released in the stomach of the subject. Following oral administration of the (Z)-endoxifen enteric-resistant, delayed release formulation, at least about 85% of endoxifen present in the blood plasma and serum of the subject is in the Z-isoform.

Oral administration of a high dose of (Z)-endoxifen eliminates, slows the spread of, shrinks, or reduces symptoms of the cervical cancer, thereby treating the cervical cancer.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a drug formulation comprising:
not less than 4% (Z)-endoxifen by weight, and
not less than 1% croscarmellose sodium by weight; and
an enteric-resistant delayed release capsule encapsulating the drug formulation.

2. The composition of claim 1, wherein a percent of (Z)-endoxifen with respect to total endoxifen in the drug formulation is not less than 95%, not less than 97%, or not less than 98%, wherein the total endoxifen consists of (Z)-endoxifen and (E)-endoxifen.

3. The composition of claim 2, wherein the percent of (Z)-endoxifen with respect to total endoxifen in the drug formulation is not more than 100%.

4. The composition of any one of claims 1-3, wherein the drug formulation comprises not less than 5%, not less than 7%, not less than 10%, not less than 12%, or not less than 15% (Z)-endoxifen by weight.

5. The composition of any one of claims 1-4, wherein the drug formulation comprises not more than 20%, not more than 22%, not more than 25%, not more than 30%, or not more than 40% (Z)-endoxifen by weight.

6. The composition of any one of claims 1-5, wherein the drug formulation comprises not less than 5% and not more than 40% (Z)-endoxifen by weight.

7. The composition of any one of claims 1-6, wherein the drug formulation comprises not less than 10% and not more than 25% (Z)-endoxifen by weight.

8. The composition of any one of claims 1-7, wherein the drug formulation comprises not less than 15% and not more than 20% (Z)-endoxifen by weight.

9. The composition of any one of claims 1-8, wherein the drug formulation comprises not less than 17% and not more than 19% (Z)-endoxifen by weight.

10. The composition of any one of claims 1-9, wherein the drug formulation comprises not less than 1 mg and not more than 80 mg (Z)-endoxifen per enteric-resistant delayed release capsule.

11. The composition of any one of claims 1-10, wherein the drug formulation comprises not less than 10 mg and not more than 80 mg (Z)-endoxifen per enteric-resistant delayed release capsule.

12. The composition of any one of claims 1-11, wherein the drug formulation comprises not less than 30 mg and not more than 50 mg (Z)-endoxifen per enteric-resistant delayed release capsule.

13. The composition of any one of claims 1-12, wherein the drug formulation comprises not less than 38 mg and not more than 42 mg (Z)-endoxifen per enteric-resistant delayed release capsule.

14. The composition of any one of claims 1-13, wherein the drug formulation comprises about 1 mg, about 2 mg, about 4 mg, about 10 mg, about 20 mg, about 40 mg, or about 80 mg (Z)-endoxifen per enteric-resistant delayed release capsule.

15. The composition of any one of claims 1-14, wherein the drug formulation comprises not less than 1.5%, not less than 1.7%, not less than 2%, not less than 2.2%, not less than 2.5%, not less than 2.7%, or not less than 2.8% croscarmellose sodium by weight.

16. The composition of any one of claims 1-15, wherein the drug formulation comprises not more than 3%, not more than 3.2%, not more than 3.5%, not more than 4%, not more than 4.5%, or not more than 5% croscarmellose sodium by weight.

17. The composition of any one of claims 1-16, wherein the drug formulation comprises not less than 1% and not more than 5% croscarmellose sodium by weight.

18. The composition of any one of claims 1-17, wherein the drug formulation comprises not less than 2% and not more than 4% croscarmellose sodium by weight.

19. The composition of any one of claims 1-18, wherein the drug formulation comprises not less than 2.5% and not more than 3% croscarmellose sodium by weight.

20. The composition of any one of claims 1-19, wherein the drug formulation comprises not less than 2.8% and not more than 3% croscarmellose sodium by weight.

21. The composition of any one of claims 1-20, wherein the drug formulation further comprises microcrystalline cellulose.

22. The composition of claim 21, wherein the drug formulation comprises not less than 60%, not less than 65%, not less than 70%, not less than 75%, or not less than 77% microcrystalline cellulose by weight.

23. The composition of claim 21 or claim 22, wherein the drug formulation comprises not more than 79%, not more than 80%, not more than 85%, not more than 90%, or not more than 95% microcrystalline cellulose by weight.

24. The composition of any one of claims 21-23, wherein the drug formulation comprises not less than 60% and not more than 95% microcrystalline cellulose by weight.

25. The composition of any one of claims 21-24, wherein the drug formulation comprises not less than 70% and not more than 90% microcrystalline cellulose by weight.

26. The composition of any one of claims 21-25, wherein the drug formulation comprises not less than 75% and not more than 80% microcrystalline cellulose by weight.

27. The composition of any one of claims 1-26, wherein the drug formulation further comprises magnesium stearate.

28. The composition of claim 27, wherein the drug formulation comprises not less than 0.3%, not less than 0.5%, not less than 0.7%, not less than 0.8%, or not less than 0.9% magnesium stearate by weight.

29. The composition of claim 27 or claim 28, wherein the drug formulation comprises not more than 1.1%, not more than 1.2%, not more than 1.5%, not more than 1.8%, not more than 2%, or not more than 3% magnesium stearate by weight.

30. The composition of any one of claims 27-29, wherein the drug formulation comprises not less than 0.5% and not more than 3% magnesium stearate by weight.

31. The composition of any one of claims 27-30, wherein the drug formulation comprises not less than 0.5% and not more than 2% magnesium stearate by weight.

32. The composition of any one of claims 27-31, wherein the drug formulation comprises not less than 0.5% and not more than 1.5% magnesium stearate by weight.

33. The composition of any one of claims 1-32, wherein the enteric-resistant delayed release capsule comprises hydroxypropyl methylcellulose, gellan gum, gelatin, hydroxypropyl methylcellulose phthalate, a coloring agent, an opacifier, or any combination thereof.

34. The composition of claim 33, wherein the enteric-resistant delayed release capsule comprises hydroxypropyl methylcellulose.

35. The composition of claim 34, wherein the enteric-resistant delayed release capsule comprises not less than 85% and not more than 97% hydroxypropyl methylcellulose by weight.

36. The composition of any one of claims 33-35, wherein the enteric-resistant delayed release capsule comprises gellan gum, gelatin, or a combination thereof.

37. The composition of claim 36, wherein the enteric-resistant delayed release capsule comprises not less than 3% and not more than 10% the gellan gum, the gelatin, or the combination thereof by weight.

38. The composition of any one of claims 1-37, wherein the (Z)-endoxifen comprises a polymorphic form of endoxifen.

39. The composition of claim 38, wherein the polymorphic form of endoxifen is Form I, characterized by an x-ray powder diffraction pattern comprising major peaks at 16.8±0.3°, 17.1±0.3° and 21.8±0.3° two theta.

40. The composition of claim 39, wherein the x-ray powder diffraction pattern further comprises:
a) at least one peak selected from 16.0±0.3°, 18.8±0.3° and 26.5±0.3° two theta;
b) at least one peak selected from 12.3±0.3°, 28.0±0.3° and 29.0±0.3° two theta;
or
c) a combination thereof.

41. The composition of claim 39, wherein the x-ray powder diffraction pattern further comprises at least one peak, or at least two peaks, at least three peaks selected from the group consisting of 16.0±0.3°, 18.8±0.3° and 26.5±0.3° two theta.

42. The composition of claim 41, wherein the x-ray powder diffraction pattern further comprises at least one peak, or at least two peaks, at least three peaks selected from the group consisting of 12.3±0.3°, 28.0±0.3° and 29.0±0.3° two theta.

43. The composition of claim 38, wherein the polymorphic form of endoxifen is Form II, characterized by an x-ray powder diffraction pattern comprising major peaks at 7.0±0.3°, 11.9±0.3°, and 14.0±0.3°.

44. The composition of claim 43, wherein the x-ray powder diffraction pattern further comprises at least one peak, or at least two peaks, at least three peaks, or at least four peaks selected from the group consisting of the group consisting of 18.4±0.3°, 22.0±0.3°, 6.6±0.3°, and 13.3±0.3° two theta.

45. The composition of claim 44, wherein the x-ray powder diffraction pattern further comprises at least one peak, or at least two peaks, at least three peaks, at least four peaks, or at least five peaks selected from the group consisting of 20.0±0.3°, 6.6±0.3°, 13.3±0.3°, 20.0±0.3° and 22.0±0.3° two theta.

46. The composition of claim 38, wherein the polymorphic form of endoxifen is Form III, characterized by an x-ray powder diffraction pattern comprising major peaks at 11.9±0.3°, 13.9±0.3°, and 17.1±0.3° two theta.

47. The composition of claim 46, wherein the x-ray powder diffraction pattern further comprises at least one peak, or at least two peaks, at least three peaks, or at least four peaks selected from the group consisting of 17.7±0.3°, 25.3±0.3°, 18.2±0.3°, and 22.5±0.3° two theta.

48. The composition of claim 47, wherein the x-ray powder diffraction pattern further comprises at least one peak, or at least two peaks, at least three peaks, at least four peaks, or at least five peaks selected from the group consisting of 26.8±0.3°, 18.2±0.3°, 22.5±0.3°, 25.3±0.3° and 26.8±0.3° two theta.

49. The composition of claim 38, wherein the polymorphic form of endoxifen is Form IV, characterized by an x-ray powder diffraction pattern comprising major peaks at 4.7±0.3° two theta, 23.3±0.3°, and 13.6±0.3° two theta.

50. The composition of claim 49, wherein the x-ray powder diffraction pattern further comprises at least one peak, at least two peaks, at least three peaks, or at least four peaks selected from the group consisting of 23.8±0.3°, 14.2±0.3°, 22.5±0.3°, or 15.7±0.3° two theta.

51. The composition of claim 40, wherein the x-ray powder diffraction pattern further comprises at least one peak, at least two peaks, or at least three peaks selected from the group consisting of 7.1±0.3°, 20.2±0.3°, or 9.5±0.3° two theta.

52. The composition of claim 38, wherein the polymorphic form of endoxifen is Form V, characterized by an x-ray powder diffraction pattern comprising major peaks at 12.5±0.3°, 19.6±0.3°, and 8.9±0.3° two theta.

53. The composition of claim 52, wherein the x-ray powder diffraction pattern further comprises at least one peak, at least two peaks, at least three peaks, or at least four peaks selected from the group consisting of 21.7±0.3°, 20.8±0.3°, 19.8±0.3°, or 16.0±0.3° two theta.

54. The composition of claim 53, wherein the x-ray powder diffraction pattern further comprises at least one peak, comprises at least two peaks, at least three peaks, at least four peaks, at least five peaks, or at least six peaks selected from the group consisting of 21.7±0.3°, 20.8±0.3°, 19.8±0.3°, 16.0±0.3°, 22.0±0.3°, 13.5±0.3°, and 14.4±0.3° two theta.

55. The composition of claim 38, wherein the polymorphic form of endoxifen is Form VI, characterized by an x-ray powder diffraction pattern comprising major peaks at 9.9±0.3°, 13.4±0.3°, and 13.7±0.3° two theta.

56. The composition of claim 55, wherein the x-ray powder diffraction pattern further comprises at least one peak, at least two peaks, at least three peaks, or at least four peaks selected from the group consisting of 17.6±0.3°, 18.6±0.3°, 17.3±0.3°, or 21.8±0.3° two theta.

57. The composition of claim 56, wherein the x-ray powder diffraction pattern further comprises at least one peak, at least two peaks, or at least three peaks selected from the group consisting of 10.2±0.3°, 19.5±0.3°, or 14.2±0.3° two theta.

58. The composition of claim 38, wherein the polymorphic form of endoxifen is Form VII, characterized by an x-ray powder diffraction pattern comprising major peaks at 20.0±0.3°, 22.6±0.3°, and 10.6±0.3° two theta.

59. The composition of claim 58, wherein the x-ray powder diffraction pattern further comprises at least one peak, at least two peaks, at least three peaks, or at least four peaks selected from the group consisting of 11.4±0.3°, 16.4±0.3°, 9.6±0.3°, or 13.3±0.3° two theta.

60. The composition of claim 59, wherein the x-ray powder diffraction pattern further comprises at least one peak, at least two peaks, or at least three peaks selected from the group consisting of 18.2±0.3°, 13.1±0.3°, or 27.0±0.3° two theta.

61. The composition of claim 38, wherein the polymorphic form of endoxifen is Form VIII, characterized by an x-ray powder diffraction pattern comprising major peaks at 4.8±0.3°, 18.9±0.3°, and 9.5±0.3° two theta.

62. The composition of claim 61, wherein the x-ray powder diffraction pattern further comprises at least one peak, at least two peaks, at least three peaks, or at least four peaks selected from the group consisting of 23.7±0.3°, 21.9±0.3°, 21.2±0.3°, or 12.9±0.3° two theta.

63. The composition of claim 62, wherein the x-ray powder diffraction pattern further comprises at least one peak, at least two peaks, or at least three peaks selected from the group consisting of 25.0±0.3°, 21.5±0.3°, or 16.4±0.3° two theta.

64. The composition of claim 38, wherein the polymorphic form of endoxifen is Form IX, characterized by an x-ray powder diffraction pattern comprising major peaks at 19.0±0.3°, 12.9±0.3°, and 15.9±0.3° two theta.

65. The composition of claim 64, wherein the x-ray powder diffraction pattern further comprises at least two peaks, at least three peaks, or at least four peaks selected from the group consisting of 21.7±0.3°, 20.8±0.3°, 21.1±0.3°, or 8.9±0.3° two theta.

66. The composition of claim 65, wherein the x-ray powder diffraction pattern further comprises at least one peak, at least two peaks, or at least three peaks selected from the group consisting of 16.4±0.3°, 4.2±0.3°, or 12.7±0.3° two theta.

67. The composition of claim 38, wherein the polymorphic form of endoxifen is Form X, characterized by an x-ray powder diffraction pattern comprising major peaks at 7.2±0.3°, 14.3±0.3°, 18.7±0.3°, and two theta.

68. The composition of claim 67, wherein the x-ray powder diffraction pattern further comprises at least one peak, at least two peaks, or at least three peaks selected from the group consisting of 21.5±0.3°, and 22.7±0.3°, and 17.1±0.3° two theta.

69. The composition of claim 68, wherein the x-ray powder diffraction pattern further comprises at least one peak, at least two peaks, or at least three peaks selected from the group consisting of 21.8±0.3°, 27.3±0.3°, or 29.4±0.3° two theta.

70. The composition of claim 38, wherein the polymorphic form of endoxifen is Form XI, characterized by an x-ray powder diffraction pattern comprising major peaks at 14.0±0.3°, 17.7±0.3°, and 11.9±0.3° two theta.

71. The composition of claim 70, wherein the x-ray powder diffraction pattern further comprises at least one peak, at least two peaks, or at least three peaks selected from the group consisting of 18.4±0.3°, 23.9±0.3°, or 17.3±0.3° two theta.

72. The composition of claim 71, wherein the x-ray powder diffraction pattern further comprises at least one peak, at least two peaks, at least three peaks, or at least four peaks selected from the group consisting of 21.8±0.3°, 20.8±0.3°, and 23.0±0.3°, or 22.2±0.3° two theta.

73. The composition of claim 38, wherein the polymorphic form of endoxifen is Form XII, characterized by an x-ray powder diffraction pattern comprising major peaks at 12.5±0.3°, 15.6±0.3°, and 19.0±0.3° two theta.

74. The composition of claim 73, wherein the x-ray powder diffraction pattern further comprises at least one peak, at least two peaks, at least three peaks, or at least four peaks selected from the group consisting of 21.9±0.3°, 20.2±0.3°, 16.0±0.3°, or 21.6±0.3° two theta.

75. The composition of claim 74, wherein the x-ray powder diffraction pattern further comprises at least one peak, at least two peaks, or at least three peaks selected from the group consisting of 22.4±0.3°, 16.8±0.3°, or 12.8±0.3° two theta.

76. The composition of claim 38, wherein the polymorphic form of endoxifen is Form XIV, characterized by an x-ray powder diffraction pattern comprising major peaks at 11.6±0.3°, 21.3±0.3°, and 19.3±0.3° two theta.

77. The composition of claim 76, wherein the x-ray powder diffraction pattern further comprises at least one peak, at least two peaks, at least three peaks, or at least four peaks selected from the group consisting of 17.5±0.3°, 15.4±0.3°, 21.6±0.3°, or 5.8±0.3° two theta.

78. The composition of claim 77, wherein the x-ray powder diffraction pattern further comprises at least one peak, at least two peaks, or at least three peaks selected from the group consisting of 16.3±0.3°, 21.9±0.3°, or 23.9±0.3° two theta.

79. The composition of claim 38, wherein the polymorphic form of endoxifen is Form XV, characterized by an x-ray powder diffraction pattern comprising major peaks at 9.8±0.3°, 4.7±0.3°, and 14.0±0.3° two theta.

80. The composition of claim 79, wherein the x-ray powder diffraction pattern further comprises at least one peak, at least two peaks, at least three peaks, or at least four peaks selected from the group consisting of 20.2±0.3°, 7.1±0.3°, 23.4±0.3°, or 22.4±0.3° two theta.

81. The composition of claim 80, wherein the x-ray powder diffraction pattern further comprises at least one peak, at least two peaks, or at least three peaks selected from the group consisting of 21.7±0.3°, 22.7±0.3°, or 18.8±0.3° two theta.

82. The composition of claim 38, wherein the polymorphic form of endoxifen is Form XIX, characterized by an x-ray powder diffraction pattern comprising major peaks at 4.7±0.3°, 23.6±0.3°, and 18.9±0.3° two theta.

83. The composition of claim 82, wherein the x-ray powder diffraction pattern further comprises at least one peak, at least two peaks, at least three peaks, or at least four peaks selected from the group consisting of 9.4±0.3°, 23.3±0.3°, 22.3±0.3°, or 20.1±0.3° two theta.

84. The composition of claim 83, wherein the x-ray powder diffraction pattern further comprises at least one peak, at least two peaks, or at least three peaks selected from the group consisting of 19.6±0.3°, 7.1±0.3°, or 15.7±0.3° two theta.

85. The composition of any one of claims 38-84, wherein at least 90% of the (Z)-endoxifen by weight is the polymorphic form of endoxifen.

86. The composition of any one of claims 1-85, comprising an in vitro dissolution profile wherein:
a) not more than 20% of the (Z)-endoxifen is released within 2 hours after the composition is introduced into an acidic stage of an in vitro dissolution assay;
b) not less than 70% of the (Z)-endoxifen is released within 1.5 hours after the composition is introduced into a buffer stage of the in vitro dissolution assay; or
c) a combination thereof.

87. The composition of claim 86, wherein not more than 5%, not more than 10%, or not more than 15% of the (Z)-endoxifen is released within 2 hours after the composition is introduced into the acidic stage of the in vitro dissolution assay.

88. The composition of claim 86 or claim 87, wherein not less than 75%, not less than 80%, or not less than 85% of the (Z)-endoxifen is released within 1.5 hours after the composition is introduced into the buffer stage of the in vitro dissolution assay.

89. The composition of any one of claims 86-88, wherein the buffer stage comprises a pH of less than 2.

90. The composition of any one of claims 86-89, wherein the buffer stage comprises a pH of about 6.8.

91. The composition of any one of claims 86-90, wherein the buffer stage comprises 0.75% polysorbate 80.

92. The composition of any one of claims 1-91, wherein upon oral administration of the composition to a subject not more than 5%, not more than 10%, not more than 15%, or not more than 20% of the (Z)-endoxifen is released in a stomach of the subject, and not less than 70%, not less than 75%, not less than 80%, or not less than 85% of the (Z)-endoxifen is released in an intestine of the subject.

93. A composition comprising:
a drug formulation comprising:
not less than 15% and not more than 20% (Z)-endoxifen by weight,
not less than 2% and not more than 4% croscarmellose sodium by weight,
not less than 0.5% and not more than 2% magnesium stearate by weight, and
not less than 70% and not more than 80% microcrystalline cellulose by weight; and
an enteric-resistant delayed release capsule encapsulating the drug formulation, wherein the enteric-resistant delayed release capsule comprises:
not less than 85% and not more than 97% hydroxypropyl methylcellulose by weight, and
not less than 3% and not more than 7% gellan gum by weight.

94. The composition of claim 93, wherein the drug formulation comprises not less than 17% and not more than 19% (Z)-endoxifen by weight.

95. The composition of claim 93 or claim 94, wherein the drug formulation comprises not less than 2.8% and not more than 3% croscarmellose sodium by weight.

96. The composition of any one of claims 93-95, wherein the drug formulation comprises not less than 0.7% and not more than 1.2% magnesium stearate by weight.

97. The composition of any one of claims 93-96, wherein the drug formulation comprises not less than 75% and not more than 80% microcrystalline cellulose by weight.

98. A method of treating a disorder in a subject, the method comprising orally administering to the subject a composition comprising:
a drug formulation comprising:
not less than 4% (Z)-endoxifen by weight, and
not less than 1% croscarmellose sodium by weight; and
an enteric-resistant delayed release capsule encapsulating the drug formulation thereby treating the cancer.

99. A method of treating a disorder in a subject, the method comprising orally administering to the subject the composition of any one of claims 1-97, thereby treating the disorder.

100. The method of claim 98 or claim 99, wherein the disorder is a cancer, a hormone-dependent breast disorder, or a hormone-dependent reproductive tract disorder.

101. The method of claim 100, wherein the cancer is breast cancer, cervical cancer, ovarian cancer, endometrial cancer, uterine cancer, vaginal cancer, vulvar cancer, melanoma, colorectal cancer, gastric cancer, neuroblastoma, pancreatic cancer, esophageal cancer, rectal cancer, or cholangiocarcinoma.

102. The method of claim 101, wherein the breast cancer is triple negative breast cancer, ductal carcinoma in situ, lobular carcinoma in situ, invasive ductal carcinoma, or invasive lobular carcinoma.

103. The method of claim 100, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, precocious puberty, or McCune-Albright Syndrome.

104. The method of any one of claims 98-103, wherein the disorder is tamoxifen-resistant or tamoxifen-refractory.

105. The method of any one of claims 98-104, further comprising releasing not more than 5%, not more than 10%, not more than 15%, or not more than 20% of the (Z)-endoxifen is released in a stomach of the subject, and not less than 70%, not less than 75%, not less than 80%, or not less than 85% of the (Z)-endoxifen is released in an intestine of the subject following oral administration of the composition.

106. The method of any one of claims 98-105, further comprising producing in the subject a blood plasma concentration of endoxifen that is not less than 70%, not less than 75%, not less than 80%, or not less than 85% in the (Z)-isoform.

107. The method of any one of claims 98-106, comprising orally administering to the subject not less than 10 mg and not more than 100 mg of the (Z)-endoxifen per day.

108. The method of any one of claims 98-107, comprising orally administering to the subject not less than 30 mg and not more than 90 mg of the (Z)-endoxifen per day.
